(19) United States
(12) Patent Application Publication
(10) Pub. No.: US 2026/0325531 A1
(43) Pub. Date: Oct. 22, 2026

(54) METHODS FOR REPROGRAMMING CANCER CELLS

(71) Applicant: Thomas Jefferson University, Philadelphia, PA (US)

(72) Inventors: Alexander Mazo, Cherry Hill, NJ (US); Bruno Calabretta, Philadelphia, PA (US); Svetlana Petruk, Voorhees, NJ (US); Patrizia Porazzi, Philadelphia, PA (US); David Deming, Philadelphia, PA (US)

(21) Appl. No.: 19/726,351

(22) Filed: Jun. 30, 2026

Related U.S. Application Data
(63) Continuation of application No. 17/636,997, filed on Aug. 21, 2020.
(60) Provisional application No. 62/890,301, filed on Aug. 22, 2019.

(51) Int. Cl. A61K 31/5377; A61K 31/42; A61K 31/496; A61K 31/519; A61K 31/573; A61P 35/02

(52) U.S. Cl. CPC A61K 31/5377; A61K 31/42; A61K 31/519; A61K 31/573; A61P 35/02

(57) ABSTRACT

The present invention relates to methods and compositions for the treatment of cancer in a subject in need thereof by treatments that reprogram the cancer cells.

### CROSS REFERENCE TO RELATED APPLICATION

The present application is a continuation of, and claims priority to, Ser. No. 17/636,997, filed Feb. 21, 2022, now allowed, which is a 35 U.S.C. § 371 national phase application from, and claims priority to, International Application No. PCT/US2020/047471, filed Aug. 21, 2020, and published under PCT Article 21(2) in English, which claims priority under 35 U.S.C. § 119(e) to U.S. Provisional Patent Application No. 62/890,301 filed Aug. 22, 2019, all of which applications are incorporated herein by reference in their entireties.

### STATEMENT REGARDING FEDERALLY SPONSORED RESEARCH OR DEVELOPMENT

This invention was made with government support under HL127895 and AI12565001 awarded by the National Institutes of Health. The government has certain rights in the invention.

### BACKGROUND OF THE INVENTION

Leukemia results from transformation of early hematopoietic progenitor cells (HPCs)/stem cells, leading to differentiation arrest and significant alterations in transcriptional and epigenetic programs. During lineage choice, multipotent HPCs undergo global changes in gene expression that correlate with the association of positive or negative histone marks with genomic DNA (e.g., H3K4me3/H3K27Ac or H3K27me3/). Chromatin structure is important for recruitment of lineage-determining transcription factor (TFs) proteins to the regulatory regions of genes essential for differentiation. In particular, TFs must overcome the barrier of condensed H3K27me3-marked chromatin, which associates with all repressed genes in the genome, to gain access to their DNA-binding sites.

One shared hallmark of a large subset of hematologic malignancies is the inability of the neoplastic cells to proceed through normal differentiation. The maturation-arrest holds leukemia cells at an immature and self-renewing stage of development and ensures continuous expansion of genetically and epigenetically unstable leukemic clones driving disease progression and relapse. Differentiation therapy is a treatment strategy in which leukemia cells are induced to express TFs that drive their differentiation into mature blood cells, halting proliferation and causing cell cycle arrest, senescence, and apoptosis. However, attempts to apply differentiation therapy to most types of leukemia such as acute myeloid leukemia (AML) have largely failed, potentially due to the inability of induced TFs to bind to condensed chromatin of repressed genes.

Thus, a need exists to improve methods of reprograming cancer cells in order to treat hematologic cancers like AML. The present invention addresses this need.

### SUMMARY OF THE INVENTION

As described herein, the present invention relates to compositions and methods for the treatment of cancer in a subject in need thereof by treatments that reprogram the cancer cells.

In one aspect, the invention includes a method of treating cancer in a subject. The method comprises administering to the subject an effective amount of a histone methyltransferase inhibitor, and an effective amount of a reprogramming agent.

In certain embodiments, the method further comprises administering to the subject an effective amount of a cell cycle inhibitor. In certain embodiments, the cell cycle inhibitor is an inhibitor of any one or both of CDK4 and CDK6. In certain embodiments, the CDK4 and CDK6 inhibitor is selected from the group consisting of abemaciclib, palbociclib, and ribociclib.

In certain embodiments, the cell cycle inhibitor is administered first, followed by the histone methyltransferase inhibitor, followed by the reprogramming agent. In certain embodiments, the cell cycle inhibitor, histone methyltransferase inhibitor, and reprogramming agent are administered concurrently. In certain embodiments, the cell cycle inhibitor is administered first, followed by concurrent administration of the histone methyltransferase inhibitor and reprogramming agent.

In certain embodiments, the histone methyltransferase inhibitor is an inhibitor of the methylation of H3K27. In certain embodiments, the histone methyltransferase inhibitor is an inhibitor of enzyme Enhancer of Zeste Homolog 2 (EZH2). In certain embodiments, the EZH2 inhibitor is selected from the group consisting of tazemetostat, GSK126, 3-deazaneplanocin A, and GSK343.

In certain embodiments, the reprogramming agent induces the terminal differentiation of hematopoietic cells. In certain embodiments, the reprogramming agent is all-trans retinoic acid (ATRA). In certain embodiments, the reprogramming agent is ISX-9. In certain embodiments, the reprogramming agent is dexamethasone. In certain embodiments, the reprogramming agent is a vitamin D receptor agonist. In certain embodiments, the vitamin D receptor agonist is selected from the group consisting of 1,25 dihydroxy vitamin D3, paricalcitol, and doxercalciferol. In certain embodiments, the reprogramming agent is a peroxisome proliferator activated receptor gamma (PPARγ) receptor ligand. In certain embodiments, the PPARγ receptor ligand is a thiazolidinedione (TZD). In certain embodiments, the thiazolidinedione is selected from the group consisting of rosiglitazone, pioglitazone, metformin, or any combination thereof.

In certain embodiments, the subject is a human.

In certain embodiments, the cancer is a hematologic malignancy. In certain embodiments, the hematologic malignancy is selected from the group consisting of acute myelogenous leukemia, acute myeloblastic leukemia, acute myeloid leukemia, chronic myelogenous leukemia-blast crisis, and acute nonlymphocytic leukemia.

In another aspect, the invention includes a method of treating cancer in a subject comprising: a) administering to a sample from the subject, a plurality of combinations of histone methyltransferase inhibitors and reprogramming agents, b) determining the optimal combination of histone methyltransferase inhibitors and reprogramming agents, and c) treating the subject with the optimal combination of histone methyltransferase inhibitors and reprogramming agents.

In certain embodiments, the histone methyltransferase inhibitor is an inhibitor of the methylation of H3K27. In certain embodiments, the histone methyltransferase inhibitor is an inhibitor of enzyme Enhancer of Zeste Homolog 2 (EZH2). In certain embodiments, the EZH2 inhibitor is selected from the group consisting of tazemetostat, GSK126, 3-deazaneplanocin A, and GSK343.

In certain embodiments, the reprogramming agent induces the terminal differentiation of hematopoietic cells. In certain embodiments, the reprogramming agent t is all-trans retinoic acid (ATRA). In certain embodiments, the reprogramming agent is ISX-9. In certain embodiments, the reprogramming agent is dexamethasone. In certain embodiments, the reprogramming agent is a vitamin D receptor agonist. In certain embodiments, the vitamin D receptor agonist is selected from the group consisting of 1,25 dihydroxy vitamin D3, paricalcitol, and doxercalciferol. In certain embodiments, the reprogramming agent is a peroxisome proliferator activated receptor gamma (PPARγ) receptor ligand. In certain embodiments, the PPARγ receptor ligand is a thiazolidinedione (TZD). In certain embodiments, the thiazolidinedione is selected from the group consisting of rosiglitazone, pioglitazone, metformin, or any combination thereof.

In certain embodiments, the subject is a human.

In certain embodiments, the cancer is a hematologic malignancy. In certain embodiments, the hematologic malignancy is selected from the group consisting of acute myelogenous leukemia, acute myeloblastic leukemia, acute myeloid leukemia, chronic myelogenous leukemia-blast crisis, and acute nonlymphocytic leukemia.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1B illustrate the re-ChIP and chromatin assembly (CAA) assays. FIG. 1A is a schematic diagram of the CAA assay. DNA is labeled with EdU in vivo. EdU and conjugated biotin are shown as light and dark circles on nascent DNA. Examined proteins are shown as squares. Proximity ligation assay (PLA) signals are shown, linking the anti-biotin and anti-query antibodies. Following PLA, cells can be immunostained with antibodies to Biotin ab (EdU), brdU, and cell differentiation markers. FIG. 1B is a schematic diagram of re-ChIP with brdU. DNA is labeled with brdU in vivo, chromatin is cross-linked, sonicated, and immunoprecipitated (IPed) first with Ab to tested protein, and then IPed with Ab to brdU.

FIGS. 2A-2D illustrate H3K27me3 accumulation in hematopoietic progenitor cells (HPCs). FIG. 2A illustrates accumulation of H3K27me3 on nascent DNA of G-CSF-mobilized CD34⁺CD38⁺HPCs. DNA was labeled with EdU for 15 min and chased to 1, 2, and 4 hr. Following conjugation with biotin, CAA was performed between nascent DNA (biotin) and H3K27me3. FIG. 2B is a quantification of the results of CAA experiments shown in FIG. 2A by counting the number of PLA signals per EdU-labeled nuclei in 50 cells/each of the three independent experiments. Error bars represent +/−standard deviation, p-values were determined by ANOVA. *, p<0.05. FIG. 2C shows that treatment of CD34⁺CD38⁻HPCs with G-CSF leads to a delay in accumulation of H3K27me3 on nascent DNA at 12 hours following induction of differentiation. Purified CD34⁺CD38⁻HPCs were induced with G-CSF for 0, 12, and 24 hr. Cells were labeled with EdU for 15 min. CAA was performed between nascent DNA (biotin) and H3K27me3. Lower panels show PLA signals only. FIG. 2D is a graph depicting quantification of the results of CAA experiments shown in FIG. 2C by counting the number of PLA signals per EdU-labeled nuclei in 50 cells/each of the three independent experiments. Error bars represent ±standard deviation, p-values were determined by ANOVA. *, p<0.05.

FIGS. 3A-3D illustrate the accumulation of H3K27me3 on nascent DNA in naïve T cells. In FIG. 3A, purified naive T cells were primed by exposure to anti-CD3-TCR and anti-CD28 for 16 hr, and further induced by addition of either IL 12+INFγ (FIG. 3B., Th1 pathway) or IL2+IL4 (FIG. 3C., Th2 pathway) for indicated times. Cells were labeled with EdU for 15 min. In FIG. 3D, cells were induced to Th1 and Th2 pathways for 8 hr. Cells were labeled with EdU for 15 min (left) and chased to 30 and 60 min. CAA was performed between nascent DNA (biotin ab) and antibody to H3K27me3. Lower panels show signals only.

FIG. 4 illustrates the differentiation of CD34⁺ HPCs. G-CSF-mobilized CD34⁺ HPCs were induced toward myeloid or erythroid differentiation by treatment with G-CSF, M-CSF, or EPO respectively, for 6, 12 and 24 hr. DNA was pulse-labeled with EdU for 15 min and chased to 1 hr. CAA was performed between nascent DNA (biotin) and C/EBPa, PU.1, or GATA-1. The graph shows quantification of the results of CAA experiments by counting the number of PLA signals per EdU-labeled nuclei in 50 cells/each of the three independent experiments. Error bars represent +/−standard deviation, p Values were determined by ANOVA. *, p<0.05.

FIGS. 5A-5B illustrate that fast accumulation of post-replicative H3K27me3 leads to decreased recruitment of lineage-determining TFs to DNA. In FIG. 5A, CD34⁺ cells were induced toward myeloid differentiation with G-CSF (left) or M-CSF (middle), or erythroid differentiation with EPO (right) for 12 hr. During the induction period cells were left untreated (upper panels) or treated with the GSKJ4 inhibitor of the H3K27me3 de-methylases (KDMs) UTX and JMJD3 (lower panels). DNA was pulse-labeled with EdU for 15 min and chased to 1 hr. CAA was performed between nascent DNA (biotin) and H3K27me3 (left columns) or (in the right columns) C/EBPa (left), PU.1 (middle) or GATA-1 (right). Lower panel shows PLA signals only. In FIG. 5B, quantification of the results of CAA experiments was determined by counting the number of PLA signals per EdU-labeled nuclei in 50 cells/each of the three independent experiments.

FIG. 6 illustrates that fast accumulation of post-replicative H3K27me3 leads to decreased recruitment of lineage-determining TFs to DNA during differentiation of T cells. Naive T cells were primed for 12 hr and stimulated to Th1 (left, middle) or Th2 (right) pathways. Cells were untreated or treated with 10 μM of GSKJ4 for 5 hr, labeled with EdU for 15 min (left) or pulse-labeled for 1 hr (middle and right). CAA was performed with antibodies to H3K27me3 (left), T-bet (middle) and GATA3 (right).

FIGS. 7A-7B illustrate that binding of C/EBPa during induction of differentiation of CD34⁺ HPCs depends on DNA replication. In FIG. 7A, CD34⁺ HPCs were labeled with EdU for 30 min and induced with G-CSF for 24 hr in the absence (Cont) or presence of 2.5 mM thymidine. 0, 2 and 4 hr after release from thymidine block, CAA was performed between nascent DNA (biotin) and C/EBPa at. Lower panel shows PLA signals only. The graph in FIG. 7B shows quantification of the results of CAA experiments by counting the number of PLA signals per EdU-labeled nuclei in 50 cells/each of the three independent experiments.

FIG. 8 illustrates that binding of T-BET during induction of differentiation of naïve T cells depends on DNA replication. Naive T cells were labeled with EdU for 30 min. Cells were primed for 12 hr and induced for 24 hr to Th1 lineage in the presence of thymidine. Thymidine block was removed for 0 hr and 3 hr. CAA was performed with T-bet antibody and immunostained for EdU.

FIG. 9 illustrates that global transient de-condensation of nascent chromatin is essential during early stages of differentiation of HSCs, HPCs, and naïve T cells.

FIGS. 10A-10C illustrate that prednisone and RXR do not affect accumulation of H3K27me3 on nascent DNA in HUT78 cells. FIG. 10A: HUT78 cells were induced with 0.2 μM of Prednisone. FIG. 10B: HUT78 cells were induced with 0.2 μM of RXR for the indicated times. Cells were labeled with EdU for 15 min, and CAA was performed between Biotin (EdU) and H3K27me3 antibody. PLA only is shown in the lower panel. FIG. 10C shows quantification of these results.

FIG. 11 illustrates a library of small molecules that was used for reprogramming of leukemic cells. This library includes ligands for nuclear hormone receptors, cytokines, ligands for GPCRs, and small molecules that were used to reprogram mature cells into pluripotent cells.

FIGS. 12A-12D illustrate the effects of the dual treatment with EZH1/2 inhibitor Tazemetostat (EPZ-6438) (EPZ) and different small molecule ligands on cell survival of various leukemic T-cell lines. Sezary syndrome (SS) cells HH (FIG. 12A) and HUT78 (FIG. 12B), and T-cell acute lymphoblastic leukemia (T-ALL) cells Jurkat E6.1 (FIG. 12C) and CCRF-HSB-2 (FIG. 12D) were grown with and without 1 μM EPZ for 24 hrs. Indicated small molecule compounds were added in indicated concentrations and cells were grown for an additional 10 days. Cell viability was assayed by flow cytometry with Sytox Green.

FIG. 13 illustrates the effects of the dual treatment with EZH1/2 inhibitor Tazemetostat (EPZ-6438) (EPZ) and different small molecule ligands on cell survival of leukemic AML cell lines THP-1, K562 and KG-1a. The assays were performed as described in FIG. 12.

FIG. 14 illustrates the results of screening for successful compounds that inhibited cell viability in the B-ALL cell lines SEM, SUP-B15, RS4;11, and BV173. Effective compounds are classified by mechanism of action or known effects on inducing pluripotency. Those that show efficacy across multiple cell lines are organized by row. Some compounds inhibited cell viability in two, three, or all four tested B-ALL cell lines.

FIGS. 15A-15C illustrate the effect of EPZ and small molecule ligand treatment on TF binding. In FIG. 15A, HUT78 cells were untreated or treated with either 1 μM of GW3965 (S3), a ligand for the LXRa, or 50 nM of prednisone, a ligand for glucocorticoid receptor (GR). Cells were immunostained with antibodies to LXRa (left), and GR (right). Nuclei stained by DAPI are shown in the lower panel. In FIG. 15B, HUT78 T-cells were untreated (Untr) or treated for 6 hr with 1 μM of S3 or 50 nM of prednisone, or pre-treated with 1 μM EPZ for 24 hr and then treated for 6 hr with 1 μM of S3, or 50 nM of prednisone. In FIG. 15C, T-ALL CCRF-HSB-2 cells were untreated or treated for 6 hr with 5 μM of ISX9, or pre-treated with 0.5 μM EPZ and then treated for 6 hr with 5 μM of ISX9. In FIG. 15B and FIG. 15C, DNA was labeled with EdU for 15 min. In FIG. 15B, CAA was performed with antibodies to H3K27me3 (left), LXRa (middle), and GR (right). In FIG. 15C, with antibody to MEF-2. PLA signals are shown in the lower panel. The schemes above show the state of condensation of nascent chromatin.

FIGS. 16A-16B illustrates the effect of synchronizing cells at the beginning of the S-phase on the efficiency of combination treatment with EZH1/2 inhibitor Tazemetostat (EPZ-6438) (EPZ) and Dexamethasone or ISX-9 on cell survival of the leukemic ALL cell line SEM. SEM cells were grown in the presence of 500 nM of the Cdk4/6 inhibitor Palbociclib and 100 μM EPZ for 24 hr. Cells were washed of Palbociclib and EPZ and grown in the presence of 100 μM EPZ for 12 hr. 10 nM Dexamethasone (FIG. 16A, DEXA) or 250 nM ISX-9 (FIG. 16B) were added, and cells were grown for additional 10 days. Cell viability was assayed by flow cytometry with Sytox Green.

FIGS. 17A-17C illustrate the effect of EZH1/2 inhibitor EPZ and Prednisone treatment on the expression of various differentiation-associated genes in THP-1 cells. FIG. 17A The AML THP-1 cells were untreated (Untr) or treated with 1 μM EPZ for 24 hr, or with 10 nM of Prednisone (Pred) for 24 hr, or with the combination of EPZ and Pred. Graphs show induction of transcription of GR target genes. FIG. 17B The B-ALL SEM cells were untreated (Untr) or treated with 1 μM EPZ for 24 hr, or with 20 nM of Dexamethasone (Dexa, top graph), or 100 nM of Prednisone (Pred, bottom graph) for 24 hr, or with the combinations of EPZ and Dexa or Pred. Graphs show induction of transcription of GR target genes. FIG. 17C The T-ALL HH cells were untreated (Untr) or treated with 1 μM EPZ for 24 hr. Top graph, cells were then treated with 50 nM of Prednisone (Pred) for 24 hr or with the combinations of EPZ and Pred. Bottom graph, cells were then treated with 1 μM of ISX-9 for 72 hr or with combinations of EPZ and ISX9. Analysis by qRT-PCR.

FIGS. 18A-18D illustrate the effects of EZH1/2 inhibition combined with several differentiation inducing compounds in patient-derived B-ALL cells. Three patient samples were grown in culture until a minimum of 10% of cells were in S-phase indicating actively replicating populations. Patient samples were then plated and treated as shown previously for SEM with or without 500 nM EPZ for 24 hours followed by treatments of Dexa, ISX9, PGE2, or Dorsomorphin with the given concentrations. All results shown are for 7 days after addition of inducers. All patient samples were cultured in Stem Cell Factor Media with a cocktail of cytokines: IL-3 (10 ng/mL), IL-6 (10 ng/mL), IL-7 (10 ng/mL), FLT-3 (20 ng/mL), Stem Cell Factor (20 ng/mL). FIG. 18A illustrates viable cell concentration per mL using Sytox Green via flowcytometry. FIGS. 18B-18C: Cells were treated essentially as in FIG. 18A. FIG. 18B illustrates Annexin V positivity via flow cytometry, FIG. 18C illustrates the same data as a normalized quantity to untreated control group. FIG. 18D illustrates cells from B-ALL patient 004 grown and treated as described in FIGS. 18A-18C. Immunophenotyping via flowcytometry for the cell surface antigens CD19 and CD22.

FIGS. 19A-19C illustrate the in vivo use of EZH1/2 inhibition in combination with Dexamethasone treatment in a mouse model of leukemia. FIG. 19A: Drug treatment protocol of NRG mice injected with SEM-1 Luc+ cells (1×10⁶/mouse). 5 days post-injection, mice were imaged and left untreated (controls), treated with GSK126 alone for 14 days (GSK126 group), or pre-treated (2 days) with GSK126 and co-treated with GSK126 and dexamethasone for 12 additional days (Dexamethasone/GSK126 group); the Dexamethasone only group was treated for 14 days, starting 7 days post SEM cell injection. FIG. 19B: Representative serial bioluminescence images of mice acquired 5 days post-injection. FIG. 19C: Top and bottom-left, representative serial bioluminescence images of mice acquired at the end of 16 day treatment (21 days post SEM cell injection). FIG. 19C, right: Graph representing fold-changes in leukemia burden (bioluminescence quantification) of mice shown in this figure.

FIGS. 20A-20C illustrate the in vivo cooperation of EZH1/2 inhibition by EPZ6438 (Tazemetostat) treatment in combination with Dexamethasone or ISX9 treatment to suppress AML growth in a mouse model of leukemia. FIG. 20A illustrates the drug treatment protocol of NRG mice injected with THP-1 Luc+ cells (1×10⁶/mouse). FIG. 20B illustrates representative serial bioluminescence images of mice acquired at the end of treatment (30 days post THP-1 cell injection) for all tested groups. FIG. 20C represents quantification of the bioluminescence fold-changes in leukemia burden of the untreated and drug-treated NRG mice after treatment (30 days post injection).

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. “About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “agonist” refers to a substance that acts like another substance and therefore stimulates an action. For example, an agonist can be a molecule capable of binding a specific protein and initiating the same reaction or activity typically produced by the binding endogenous substance. An agonist can be any molecule that increases transcription, increases translation, or increases the activity of the cognate molecule.

The term “cleavage” refers to the breakage of covalent bonds, such as in the backbone of a nucleic acid molecule. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

As used herein, the term “reprogramming agent” refers to any compound capable of functioning, directly or indirectly, as a ligand for (inducible) transcription factors. Reprogramming agents can be used in differentiation therapy and include, but are not limited to, differentiation-inducing agents.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “transgene” refers to the genetic material that has been or is about to be artificially inserted into the genome of an animal, particularly a mammal and more particularly a mammalian cell of a living animal.

To “treat” a disease as the term is used herein, means to reduce the frequency or severity of at least one sign or symptom of a disease or disorder experienced by a subject.

As used herein, the term “treatment” or “treating” encompasses prophylaxis and/or therapy. Accordingly the compositions and methods of the present invention are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

As used herein, “therapeutic index” refers to the ratio of the toxic dose, or dose of a drug that causes adverse effects incompatible with effective treatment of the disease or condition, to the effective dose, or dose of a drug that leads to the desired therapeutic effect in treatment of the disease or condition.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

Leukemia results from transformation of hematopoietic stem cells (HSCs) through acquisition of multiple mutations over time. Leukemic blasts are typically arrested at morphologically and immunophenotypically distinct stages of hematopoietic differentiation and retain the potential for uncontrolled proliferation, concomitant with significant alterations in transcriptional and epigenetic programs. In order to develop more effective therapies, it is essential to understand how these programs are maintained in early progenitors/stem cells and how they change during normal differentiation and in transformed cells. To maintain existing transcriptional programs, all cells rely on epigenetic inheritance. Epigenetic inheritance relies, in part, on DNA methylation, a semi-conserved epigenetic mark, which undergoes significant changes in cancer cells. However, DNA methylation does not account for all epigenetic information, and some components of chromatin (e.g., modified histones or proteins of the Trithorax and Polycomb group families) contribute to epigenetic marking by remaining on nascent DNA after replication, and by further guiding restoration of the state of transcription.

Across the genome, one particular type of methylated histone mark termed H3K27me3 correlates with the most condensed structure of nucleosomes and with repressed transcription. Significantly, this H3K27me3 mark undergoes dynamic changes during differentiation of HSCs. Recent studies indicate that during cytokine-induced lineage commitment of normal hematopoietic progenitors (HPCs), accumulation of H3K27me3 is delayed (1-2 hours) at replicating regions of the genome, signifying a previously unknown state of transient, globally de-condensed arrays of nucleosomes. This chromatin structure of nascent DNA is permissive for transcription factor (TF) binding. By contrast, nascent chromatin of blast cells, including the CD34+ subset, from several AML, ALL and T-PLL patients is extremely compact with a rapid accumulation (within 15 minutes after DNA replication) of the H3K27me3 repressive mark. By rapidly closing off transcription factor access to the DNA, these cancer cells are able to maintain a phenotype resembling immature precursor cells that favors rapid and unchecked proliferation. As such, in some aspects, the current invention includes a method of treating cancer in subject comprising administering an effective amount of a histone methyltransferase inhibitor, and an effective amount of a reprogramming agent (e.g. differentiation-inducing agent). In certain embodiments, the method further comprises administering an effective amount of a cell cycle inhibitor.

### Histone Modification, Chromatin Structure, and Gene Expression

Long eukaryotic genomic DNA molecules are organized and packaged into dense structures collectively called chromatin by interaction with protein complexes called histones. The strands of DNA are wound around histones to form complexes called a nucleosomes in a manner similar to thread wound around a spool. Each histone complex, also called a core particle, is an octamer of two copies each of four histone proteins H2A, H2B, H3, and H4 around which 146 base pairs of DNA are wrapped in 1.67 left-handed super-helical turns. Core particles are separated by regions of linker DNA that vary in length between ~10-90 base pairs. The packaging of DNA into tight chromatin structures can affect expression of the encoded genes by physically preventing access of transcription factor and enhancer proteins to the DNA molecule. Regions of DNA undergoing active transcription are observed to have a loose, “open” structure, while less active regions feature more tightly-packed nucleosomes, often compacted into a larger chromatin structure called the 30 nm fiber. Histone proteins can themselves undergo chemical modification in the form of methylation, phosphorylation, acetylation, ubiquitination, and sumoylation, among others. These modifications can, in turn, affect the ability of the histones to assume “open” and “closed” structures, thereby affecting transcription. In this way, chemical modification of histones acts as a type of epigenetic gene regulation.

A non-limiting example of such histone modification is the addition of three methyl groups to Lysine 27 of histone H3 to form the H3K27 histone mark. This modification is associated with significant suppression of gene transcription activity, and as such is highly associated with repressed genes in the genome. H3K27me3 is unique among histone methylation marks in that there are only two known methyltransferase enzymes, EZH1 and EZH2. EZH2, or enhancer of zeste homolog 2, is a major H3K27me3 methyltransferase that is responsible for catalyzing this modification, and it is part of the PRC2 complex. In some embodiments of the current invention, a histone methyltransferase inhibitor is used in combination with a cell cycle inhibitor and a cytotoxic chemotherapy in order to treat cancer in a subject in need thereof. In some embodiments, the histone methyltransferase inhibitor is an inhibitor of the methylation of any one or both of EZH1 and EZH2. In one embodiment, the histone methyltransferase enzyme which is the target of inhibition is EZH2. In some embodiments, the histone methyltransferase inhibitor is an inhibitor of the methylation of any one or both of H3K27 and H3K9.

That a single methyltransferase enzyme is mainly responsible for regulation of such a key repressor of gene expression makes EZH2 an attractive target for small-molecule inhibitor development. Examples of EZH2 inhibitors include but are not limited to tazemetostat (also called EPZ-6438 or EPZ), GSK126, 3-deazaneplanocin A (DZNep), EPZ005687, CPI-1205, and GSK343.

DZNep inhibits the hydrolysis of S-adenosyl-L-homocysteine (SAH), which is a product-based inhibitor of all protein methyltransferases, leading to increased cellular concentrations of SAH which in turn inhibits EZH2. However, DZNep is not specific to EZH2 and also inhibits other DNA methyltransferases.

EPZ005687, an S-adenosylmethionine (SAM) competitive inhibitor that is more selective than DZNep; it has a 50-fold increase in selectivity for EZH2 compared to EZH1. The drug blocks EZH2 activity by binding to the SET domain active site of the enzyme. EPZ005687 can also inhibit the Y641 and A677 mutants of EZH2, which may be applicable for treating non-Hodgkin's lymphoma. In 2013, Epizyme began Phase I clinical trials with another EZH2 inhibitor, tazemetostat (EPZ-6438), for patients with B-cell lymphoma. In 2020, tazemetostat, with the tradename Tazverik, was FDA approved for the treatment of metastatic or locally advanced epithelioid sarcoma and was approved for the treatment of patients with relapsed follicular lymphoma later that year. Tazemetostat exhibits a 35-fold selectivity compared to EZH1 and a >4,500 fold selectivity relative to other histone methyltransferases.

GSK126 is an S-adenosylmethionine (SAM) competitor, and is highly selective for the EZH2 enzyme with a Ki value of ~0.5 nM. In one embodiment of the current invention, the EZH2 inhibitor is GSK126.

In some embodiments, the histone methyltransferase inhibitor is an inhibitor of the tri-methylation of H3K27. In some embodiments, the histone methyltransferase inhibitor is an inhibitors of the enzymes Enhancer of Zeste Homolog 1 (EZH1) and Enhancer of Zeste Homolog 2 (EZH2). Examples of EZH1/2 inhibitors include but are not limited to tazemetostat, GSK126, 3-deazaneplanocin A, and GSK343. In one embodiment, the EZH1/2 inhibitor is GSK126.

### Cell Cycle Inhibitors

The regulation of the cell cycle is governed and controlled by specific proteins, which are regulated mainly through phosphorylation/dephosphorylation processes in a precisely timed manners. The key proteins that coordinate the initiation, progression, and completion of cell-cycle programs are cyclin dependent kinases (CDKs). Cyclin-dependent kinases belong to the serine-threonine protein kinase family, and are heterodimeric complexes composed of a catalytic kinase subunit and a regulatory cyclin subunit. CDK activity is controlled by association with their corresponding regulatory subunits (cyclins) and other CDK inhibitor proteins (Cip & Kip proteins, INK4, among others), by their phosphorylation state, and by ubiquitin-mediated proteolytic degradation (see D. G. Johnson, C. L. Walker, Annu. Rev. Pharmacol. Toxicol 39 (1999) 295-312; D. O. Morgan, Annu. Rev. Cell Dev. Biol. 13(1997) 261-291; C. J. Sherr, Science 274 (1996) 1672-1677; T. Shimamura et al., Bioorg. Med. Chem. Lett. 16 (2006) 3751-3754).

There are four CDKs that are significantly involved in controlling cellular proliferation: CDK1, which predominantly regulates the transition from the second growth phase (G2) to the mitosis phase of active cell division (M phase), and CDK2, CDK4, and CDK6, which regulate the transition from the first growth phase (G1) to the DNA synthesis phase (S phase). In early to mid G1 phase, when the cell is responsive to differentiation-inducing mitogenic stimuli, activation of CDK4-cyclin D and CDK6-cyclin D induces phosphorylation of the retinoblastoma protein (pRb). Phosphorylation of pRb releases the transcription factor E2F, which enters the nucleus and activates transcription of other cyclins which promote further progression of the cell cycle (see J. A. Diehl, Cancer Biol. Ther. 1 (2002) 226-231; C. J. Sherr, Cell 73 (1993) 1059-1065). CDK4 and CDK6 are closely related proteins with basically indistinguishable biochemical properties (M. Malumbres, M. Barbacid, Trends Biochem. Sci. 30 (2005) 630-641).

In certain embodiments of the current invention, the cell cycle inhibitor is an inhibitor of both CDK4 and CDK6. A number of CDK 4/6 inhibitors have been identified, including specific pyrido[2,3-d]pyrimidines, 2-anilinopyrimidines, diaryl ureas, benzoyl-2,4-diaminothiazoles, indolo[6,7-a]pyrrolo[3,4-c]carbazoles, and oxindoles. For example, 6-acetyl-8-cyclopentyl-5-methyl-2-(5-piperazin-1-yl-pyridin-2-ylammino)-8H-pyrido-[2,3-d]-pyrimidin-7-one (PD0332991), also called palbociclib, is currently FDA approved for estrogen receptor positive (ER+) advanced breast cancer and hormone receptor positive (HR+), HER2 negative advanced or metastatic breast cancer. Examples of other CDK4/6 inhibitors include, but are not limited to abemaciclib (LY2835219), ribociclib (LEE011), and iodine-containing pyrido[2,3-d]pyrimidine-7-one (CKIA).

### Differentiation Therapy for Cancer

The term differentiation-inducing agent or reprogramming agent, as used herein, refers to any small molecule which is known to activate, directly or indirectly, ligand-dependent transcription factors (TFs), so that these TFs can bind to repressed genes and activate transcription. However, in many cases, the activation of TFs by the inducers alone is not sufficient to drive reprogramming or differentiation, since TFs cannot bind to condensed chromatin at regulatory regions of target genes. Therefore, in certain aspects of the current invention, H3K27me3-marked condensed chromatin is de-condensed, thus allowing TFs to bind to target sites and induce new transcriptional programs, causing cell reprogramming, loss of cell viability and apoptosis.

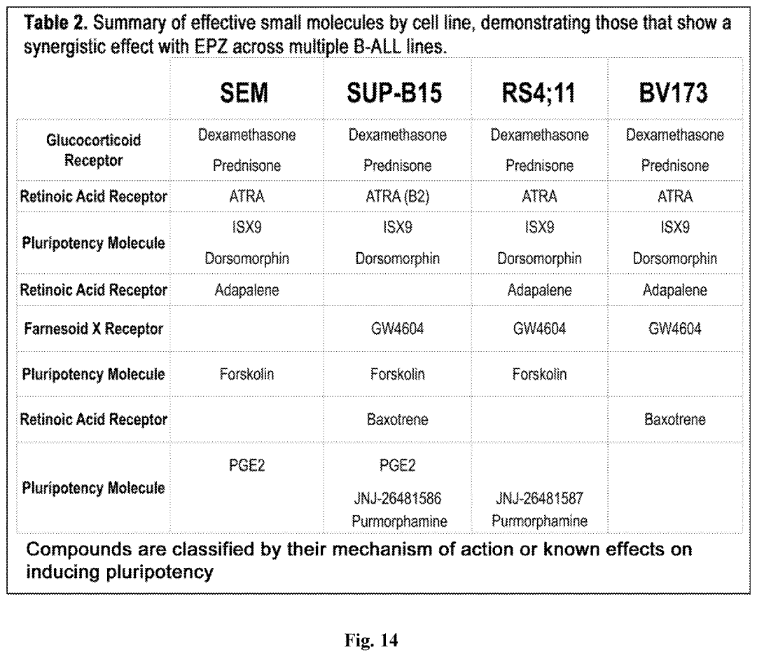

In some aspects, the current invention includes differentiation-inducing or reprogramming agents that induce the reprogramming of leukemic cells. In some embodiments, the reprogramming inducing agents belong to different groups of small molecules that serve as ligands to activate transcription factors. These include nuclear hormone receptors, pluripotency-inducing molecules, cytokines, and GPCRs (FIG. 11). Agents that induced reprogramming in several B-ALL cell lines are listed in FIG. 14.

A hallmark abnormality of many cancers is the arrest of differentiation at an early stage of development and the failure to fully mature into functional cells. By maintaining a more stem-cell like state, these cancers are able to be both highly proliferative and resistant to cytotoxic therapy. These observations of the poor differentiation status of many cancers has led to the idea of inducing differentiation/reprogramming as a form of cancer therapy. While traditional chemotherapies seek to kill tumor cells by inducing cell death, differentiation/reprogramming therapy seeks to either reactivate differentiation genetic programs in cancer cells or to reprogram them into less mature or, in some cases, an aberrant state. Reprogramming can also be accompanied by apoptosis. The reprogrammed cells may then be subjected to growth arrest or apoptosis resulting in an overall reduction in cancer progression and often in remission. Generally, reprogramming inducing agents exhibit less toxicity than cytotoxic chemotherapies.

The potential for differentiating therapy to improve cure rates in cancer is exemplified by the development of the retinoic acid receptor agonist all-trans retinoic acid (ATRA) and arsenic trioxide (ATO) for the targeted treatment of acute promyelocytic leukemia (APL). ATRA induces terminal cell differentiation by disrupting the promyelocytic leukemia/retinoic acid receptor α (PML/RARα) fusion protein that arrests the maturation of myeloid cells at the promyelocytic stage, while ATO has been shown to cause degradation of PML-RAR alpha, promoting differentiation. Pre-clincal in vitro studies found that treating HL-60 cells, an APL cell line, with either ATRA or ATO produced terminal differentiation in around 90% of cells. Subsequent experiments determined that ATRA was specifically effective in APL cells carrying a typical chromosomal translocation between chromosomes 15 and 17 but not in other leukemias. Based on these results, and successful clinical trials, ATRA and ATO have become standard of care for the treatment of APL in younger adult, non-high-risk patients. Despite these advances, APL continues to have an approximately 10% mortality rate, and 20% to 30% of patients eventually become resistant, suggesting a need for further improvements in differentiation therapy, particularly for treatment of other poorly-differentiated solid and liquid tumors. In certain embodiments of the current invention, the reprogramming agent is all-trans retinoic acid (ATRA). In certain embodiments, the reprogramming agent is a retinoic acid receptor agonist. In certain embodiments, the reprogramming agent is adapalene.

Other reprogramming agents or differentiation-inducing agents are well-known in the art, including those that act as ligands of the peroxisome proliferator activated receptor gamma (PPARγ) receptor. PPARγ is a multifunctional transcription factor with important regulatory roles in inflammation, cellular growth, differentiation, and apoptosis. PPARγ is expressed in a variety of immune cells as well as in numerous leukemias and lymphomas. When PPARγ is activated it forms a heterodimer with the nuclear hormone receptor, RXR. This nuclear hormone complex can then bind to the peroxisome proliferator response element (PPRE) and induce transactivation of many differentiation-associated genes.

A number of activating ligands of PPARγ have been identified in previous studies, including those of the class of drugs called thiazolidinediones (TZD). Also known as glitazones, after ciglitazone, the prototypical member of the class. These compounds are heterocyclic molecules initially developed for the treatment of diabetes mellitus type 2 in the 1990s. Members of the TZD class of compounds include, but are not limited to pioglitazone (Actos), rosiglitazone (Avandia), lobeglitazaone (Duvie), darglitazone, englitazone, netoglitazone, rivoglitazone, troglitazone (Rezulin), balaglitazone (DRF-2593), and the combination of rosiglitazone and the biguanide metformin. In certain embodiments, the current invention includes a PPARγ receptor ligand as a reprogramming agent. In certain embodiments, the PPARγ receptor is a thiazolidinedione (TZD). In certain embodiments, the thiazolidinedione is selected from the group consisting of rosiglitazone, pioglitazone, and the combination of rosiglitazone and metformin.

In certain embodiments, the current invention includes reprogramming agents (e.g. differentiation-inducing agents) that can influence the pluripotency of stem cells. Examples of such agents include, but are not limited to forskolin, ISX9, CHIR99021, Smoothened Agonist (SAG), Dorsomorphin, GO6983, RN-486, LDN-193189, SB-431542, Purmorphamine, LMK235, and JNJ-26481585, among others.

In a preferred embodiment, the reprogramming agent is ISX-9. ISX-9 is a small molecule inhibitor that has been commonly studied as an inducer of stem cell differentiation of multiple cell types including adult neural stem cells in both in vivo and in vitro models. ISX-9 has been shown to act through a calcium-activated signaling pathway dependent on genes expressed through monocyte-enhancer factor 2 (MEF2) dependent pathways.

In certain embodiments of the current invention, the reprogramming agent is a vitamin D receptor (VDR) agonist. These molecules are well known for their ability to regulate calcium and bone metabolism, as well as the differentiation of multiple cell types. During activation of the VDR, calcitriol, the active form of vitamin D, binds to the receptor and forms a heterodimer with the retinoid-X receptor (RXR), which then binds to hormone response elements in various gene promoter regions, resulting in expression or transrepression of specific differentiation-associated genes. Additionally, activation of the VDR also regulates micro-RNA (miRNA)-associated post-transcriptional mechanisms. Members of the VDR agonist family included in certain embodiments of the current invention include, but are not limited to, 1,25 dihydroxy vitamin D3, paricalcitol, and doxercalciferol.

In certain embodiments of the current invention, the reprogramming agent may be a glucocorticoid receptor agonist. Non-limiting examples of glucocorticoid receptor agonists are Dexamethasone, Prednisone, Budesomide, Halcinomide, Mometasone furoate, and Brequinar, among others.

In certain embodiments of the current invention, the reprogramming agent may be a G protein-coupled receptor agonist (GCPR). A non-limiting example of a GCPR agonist contemplated by the current invention is prostaglandin E2 (PGE2).

### Methods of Treatment

Certain aspects of the invention provide methods for treating cancer comprising administering to a subject an effective amount of a histone methyltransferase inhibitor, and an effective amount of a reprogramming agent. The method can further comprise administering to the subject an effective amount of a cell cycle inhibitor.

In some embodiments, the histone methyltransferase inhibitor is administered first, followed by the reprogramming agent. In some embodiments, the cell cycle inhibitor is administered first, followed by the histone methyltransferase inhibitor, followed by the reprogramming agent. In some embodiments, the cell cycle inhibitor is administered first, followed by concurrent administration of the histone methyltransferase inhibitor and the reprogramming agent. In some embodiments, the cell cycle inhibitor, histone methyltransferase inhibitor, and reprogramming agent are administered concurrently.

In some embodiments, the subject is a human. In some embodiments, the cancer is a hematologic malignancy. The term hematologic malignancy broadly refers to pathologic conditions which involve any of the many cell types that are of hematopoietic origin including lymphocytes and myeloid lineage cells. The diversity in the lineages and differentiation stages of hematopoietic cells results in a large number of distinct and heterogeneous tumors generally referred to as hematologic malignancies. Thus, hematologic malignancies or hematologic neoplasia affect cells and tissues of the blood, including blood, bone marrow and lymph nodes. Hematologic malignancies include both leukemias and lymphomas. The term leukemia has generally been used to define hematologic malignancies of the blood or bone marrow characterized by abnormal proliferation of leukocytes. The principal subtypes of leukemia are identified on the basis of malignancy involving lymphoid (e.g. T or B lymphocytic lineage) or myeloid (e.g. granulocytic, erythroid or megakaryocytic lineage) cells, and whether the disease is acute or chronic in onset. The term lymphoma covers a heterogeneous group of neoplasms of lymphoid tissue. Lymphomas are broadly categorized under Hodgkin lymphoma, and T-cell (T-NHL) and B-cell (B-NHL) non-Hodgkin lymphomas. A World Health Organization (WHO) classification has recently been published and diagnostic guidelines have been established based on this classification. In certain embodiments of the current invention, examples of hematologic malignancies include but are not limited to acute myelogenous leukemia and acute nonlymphocytic leukemia, such as acute megakaryoblastic leukemia and acute erythroblastic leukemia. In one embodiment, the hematologic malignancy is acute myelogenous leukemia. In one embodiment, the hematologic malignancy is chronic myelogenous leukemia-blast crisis.

In certain aspects, the invention includes a method of treating cancer in a subject, comprising: a) administering to a sample from the subject, a plurality of combinations of histone methyltransferase inhibitors and reprogramming agents (e.g. differentiation-inducing agents), b) determining the optimal combination of histone methyltransferase inhibitors and reprogramming agents, and c) treating the subject with the optimal combination of histone methyltransferase inhibitors and reprogramming agents.

Determining the optimal combination of histone methyltransferase inhibitors and reprogramming agents can comprise measuring from a tumor sample from a subject one or more parameters of cytotoxicity using assays known to one of ordinary skill in the art, for example cytotoxicity assays, cytokine assays, apoptosis assay (e.g. Annexin V), DNA damage assays, and the like and/or measuring expression of one or more reprogramming agents (e.g. differentiation-inducing agents (e.g. transcription factors, surface receptors, and the like)).

### Pharmaceutical Compositions

The dosage of a cell cycle inhibitor to be administered to a patient may be from about 0.1 to about 100 mg/m². In certain embodiments, the dosage may be from about 0.1 to about 70 mg/m². In certain embodiments, the dosage may be from about 0.1 to about 60 mg/m². In some embodiments, the dosage may be from about 0.1 to about 1, 5, 10, 15, 20, 15, 30, 35, 40, 41, 42, 43, 44, 45, 46, 47, 48, 49, 50, 51, 52, 53, 54, 55, 56, 57, 58, 59 or 60 mg/m². In some embodiments, the dosage may be about 0.1, 1, 5, 10, 15, 20, 15, 30, 35, 40, 41, 42, 43, 44, 45, 46, 47, 48, 49, 50, 51, 52, 53, 54, 55, 56, 57, 58, 59 or 60 mg/m².

The dosage of a histone methyltransferase inhibitor to be administered to a patient may be from about 0.1 to about 100 mg/m². In certain embodiments, the dosage may be from about 0.1 to about 70 mg/m². In certain embodiments, the dosage may be from about 0.1 to about 60 mg/m². In some embodiments, the dosage may be from about 0.1 to about 1, 5, 10, 15, 20, 15, 30, 35, 40, 41, 42, 43, 44, 45, 46, 47, 48, 49, 50, 51, 52, 53, 54, 55, 56, 57, 58, 59 or 60 mg/m². In some embodiments, the dosage may be about 0.1, 1, 5, 10, 15, 20, 15, 30, 35, 40, 41, 42, 43, 44, 45, 46, 47, 48, 49, 50, 51, 52, 53, 54, 55, 56, 57, 58, 59 or 60 mg/m².

The dosage of a reprogramming-inducing agent to be administered to a patient may be from about 0.1 to about 100 mg/m². In certain embodiments, the dosage may be from about 0.l to about 70 mg/m². In certain embodiments, the dosage may be from about 0.1 to about 60 mg/m². In some embodiments, the dosage may be from about 0.1 to about 1, 5, 10, 15, 20, 15, 30, 35, 40, 41, 42, 43, 44, 45, 46, 47, 48, 49, 50, 51, 52, 53, 54, 55, 56, 57, 58, 59 or 60 mg/m². In some embodiments, the dosage may be about 0.1, 1, 5, 10, 15, 20, 15, 30, 35, 40, 41, 42, 43, 44, 45, 46, 47, 48, 49, 50, 51, 52, 53, 54, 55, 56, 57, 58, 59 or 60 mg/m².

Human dosage amounts can initially be determined by extrapolating from the amount of compound used in mice, as a skilled artisan recognizes it is routine in the art to modify the dosage for humans compared to animal models. In certain embodiments it is envisioned that the dosage may include an effective amount from between about 0.001 mg compound/Kg body weight to about 100 mg compound/Kg body weight; or from about 0.05 mg/Kg body weight to about 75 mg/Kg body weight or from about 0.1 mg/Kg body weight to about 50 mg/Kg body weight; or from about 0.5 mg/Kg body weight to about 40 mg/Kg body weight; or from about 0.1 mg/Kg body weight to about 30 mg/Kg body weight; or from about 1 mg/Kg body weight to about 20 mg/Kg body weight. In other embodiments, the effective amount may be about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, or about 100 mg/Kg body weight. In other embodiments, it is envisaged that effective amounts may be in the range of about 2 mg compound to about 100 mg compound. In other embodiments, the effective amount may be about 1, 2, 3, 4, 5, 6, 7, 8, 9, 10, 11, 12, 13, 14, 15, 16, 17, 18, 19, or 20 mg per single dose. In another embodiment, the effective amount comprises less than about 2, 3, 4, 5, 6, 7, 8, 9, 10, 15, 20, 25, 30, 35, 40, 45, 50, 55, 60, 65, 70, 75, 80, 85, 90, 95 mg daily. In an exemplary embodiment, the effective amount comprises less than about 50 mg daily. Of course, the single dosage amount or daily dosage amount may be adjusted upward or downward, as is routinely done in such treatment protocols, depending on the results of the initial clinical trials and the needs of a particular patient.

Administration of the compositions of the present invention to a patient, preferably a mammal, more preferably a human, may be carried out using known procedures, at dosages and for periods of time effective to treat a disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder in the patient. Dosage regimens may be adjusted to provide the optimum therapeutic response. For example, several divided doses may be administered daily or the dose may be proportionally reduced as indicated by the exigencies of the therapeutic situation.

The precise determination of what would be considered an effective dose is based on factors individual to each subject, including their size, age, sex, weight, and condition of the particular subject. Dosages can be readily ascertained by those skilled in the art from this disclosure and the knowledge in the art.

Optionally, the methods of the invention provide for the administration of a composition of the invention to a suitable animal model to identify the dosage of the composition(s), concentration of components therein and timing of administering the composition(s), which elicit the desired biological response, e.g., prevention or reduction of a fibropolycystic disease. Such determinations do not require undue experimentation, but are routine and can be ascertained without undue experimentation.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

The biologically active agents can be conveniently provided to a subject as sterile liquid preparations, e.g., isotonic aqueous solutions, suspensions, emulsions, dispersions, or viscous compositions, which may be buffered to a selected pH. Cells and agents of the invention may be provided as liquid or viscous formulations. For some applications, liquid formations are desirable because they are convenient to administer, especially by injection. Where prolonged contact with a tissue is desired, a viscous composition may be preferred. Such compositions are formulated within the appropriate viscosity range. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions are prepared by suspending a FXR inhibitor in the required amount of the appropriate solvent with various amounts of the other ingredients, as desired. Such compositions may be in admixture with a suitable carrier, diluent, or excipient, such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells or agents present in their conditioned media.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent, such as methylcellulose. Other suitable thickening agents include, for example, xanthan gum, carboxymethyl cellulose, hydroxypropyl cellulose, carbomer, and the like. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form). Those skilled in the art will recognize that the components of the compositions should be selected to be chemically inert.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a fibropolycystic disease in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., analgesic and/or fibropolycystic disease agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans-and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral administration, particularly suitable are tablets, dragées, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Additional Administration Forms

In various embodiments, the compounds of the invention may be delivered transdermally. In various embodiments, the transdermal delivery formulation may contain one or more penetration enhancers.

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer than the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In certain embodiments of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The methods used in the experimental examples are now described.

Cell lines and culture conditions: The THP-1 leukemia line was derived from a patient with monocytic leukemia and were grown in a medium consisting of RPMI 1640+10% FBS+2 mM L-Glutamine. 1 mM sodium pyruvate, penicillin (100 units/ml) and streptomycin (100 μg/ml) were added to inhibit bacterial contamination. Cultures were maintained at cell densities in the range 2-9×10⁵ cells/ml at 37 °C, 5% CO₂. K562 is an erythroleukemia-type cell line derived from a patient with blast crisis of chronic myeloid leukemia, positive for the t(9;22) and expressing the P210 BCR-ABL protein. The cells are non-adherent and rounded and grow in standard culture media such IMDM with 10% FBS. The KG1a myeloblastic leukemia line was derived from a patient with acute myeloblastic leukemia. Cells were grown in standard medium (IMDM supplemented with 10% FBS). The HuT 78 T cell lymphoma line was derived from peripheral blood of a 50 years old male patient with Sezary syndrome. HuT 78 cells were grown in suspension in a medium consisting of IMDM with 10% FBS. The HH mature T cell line was derived from peripheral blood of a patient with aggressive cutaneous T cell leukemia/lymphoma. The cells were grown in RPMI 1640 supplemented with 10% FBS. The MOLT-4 cell line was derived from the peripheral blood of a 19 year old male with T-cell acute lymphoblastic leukemia in relapse. The cells were grown in RPMI-1640 supplemented with 10% FBS. The SUP-B15 cell line is a lymphoblastic leukemia line derived from a male 8 years old patient with Ph⁺ acute lymphoblastic leukemia. These cells were grown in Iscove medium supplemented with 10% FBS. The SEM cell line is a B-cell lymphoblastic leukemia line that was established from the peripheral blood of a 5-year-old girl in relapse with acute lymphoblastic leukaemia (ALL), and expresses CD19, CD22, and CDw75, as well as the myeloid antigens CD13, CD15, CD33 and CDw65. SEM cells were grown in IMDM supplemented with 10% FBS. The RS4;11 cell line was established from the bone marrow of a 32 year old-female with acute lymphoblastic leukemia. The 697 cell line was established from the bone marrow of a 12-year-old boy with acute lymphoblastic leukemia (cALL) at relapse in 1979. These cells express BCL2, BCL3 and MYC mRNA. The BV173 cell line was established from the peripheral blood of a 45-year-old man with chronic myeloid leukemia (CML) in blast crisis.

Inhibitors and chemotherapies: The EZH1/EZH2 inhibitor GSK126 and GSK343 were used at a concentration of 100 nM (SEM, SUP-B15) to 1-5 μM. For B-ALL cell lines, all glucocorticoid receptor agonists Dexamethasone, Prednisone, Budesomide, Halcinomide, Mometasone furoate, and Brequinar were all administered at 10 nM-100 nM concentrations. Other molecules and their concentration of administration are: all-trans retinoic acid (100 nM-500 nM), adapalene (50 nM-250nM), PGE (100 mM-1 μM), forskolin (250 nM-1 μM), ISX9 (250 nM-1 μM), CHIR99021 (250 nM-1 μM), Smoothened Agonist (SAG) (250 nM-1 μM), Dorsomorphin (25 nM-1 μM), GO6983 (250 nM-1 μM), RN-486 (250 nM-1 μM), LDN-193189 (250 nM-1 μM), SB-431542 (250 nM-1 μM), Purmorphamine (250 nM-1 μM), LMK235 (250 nM-1 μM), NJ-26481585 (250 nM-1 μM). Palbociclib is used at 250-500 nanomolar. For in vivo studies in NSG mice, GSK126 is used at 100 mg/Kg/IP/7 days. Palbociclib is used at 150 mg/Kg/IP, 3 days. Dexamethasone is used at 5-10 mg/liter in the drinking water for 7-14 days.

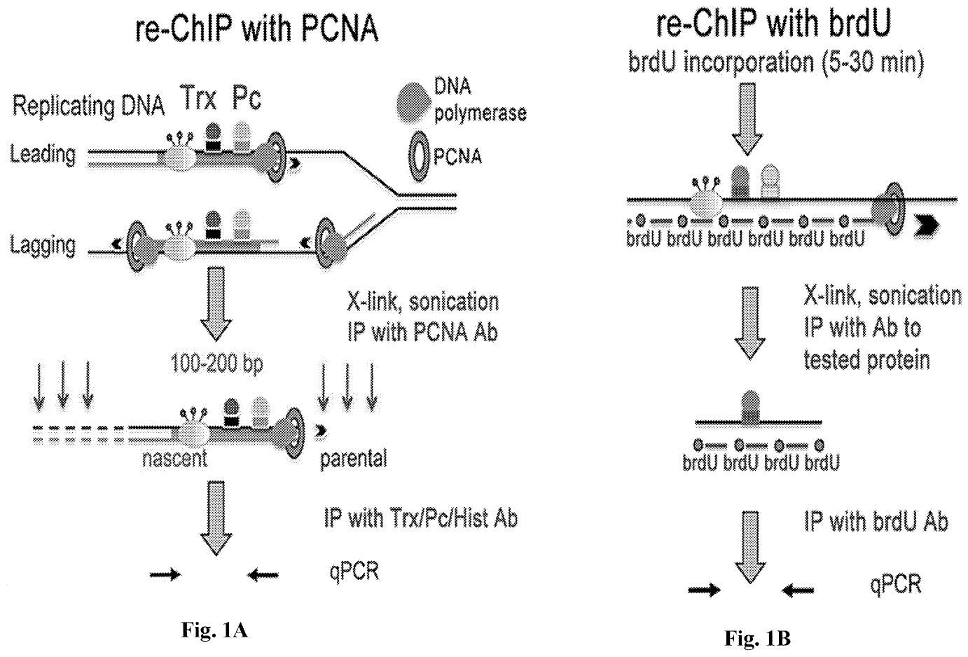

Chromatin assembly assay: Cells were grown on chamber slides, pulse-labeled with 5 μM EdU and fixed at room temperature with 4% formaldehyde in PBS for 15 min, washed with PBS, and permeabilized with 0.3% Triton for 15 min. Cells were subjected to Click-iT reaction with biotin-azide for 30 min. The PLA reactions (Olink) between the anti-biotin antibody and antibodies to other proteins were performed as described by Olink. Following PLA, cells were immunostained with anti-biotin Alexa Fluor 488 antibody to control the specificity of CAA. The results of CAA experiments shown in FIG. 1A were quantified by counting the number of PLA signals per EdU-labeled nuclei in 50 cells of each of the three independent experiments. The nature of the results in the rest of the figures alleviated the need to quantify the results of these assays.

Re-ChIP assay: 2-4×10⁷ cells were grown on 100 mm dishes, and half of the cells were induced for 2 hr 30 min with the mDA cocktail. Undifferentiated and induced cells were labeled with 50 μM BrdU for 12 min or 12 min followed by chase to 60 min, washed with PBS and fixed with 1% formaldehyde for 10 min at room temperature. After fixation glycine was added to final concentration of 0.125 M to quench formaldehyde. Cells were washed with PBS, scraped from plates, collected by centrifugation for 5 min at 1000 rpm at 4 °C, re-suspended in 1 ml of RIPA buffer (1% Triton X-100, 0.1% sodium deoxycholate, 0.05% SDS, 0.15 M NaCl, 10 mM Tris-HCl, pH 8.0, 1 mM EDTA, pH 8.0) with protease inhibitor. Suspension was sonicated to shear DNA to an average length of about 500-1000 bp and centrifuged for 5 min at 14,000 rpm. The supernatant was pre-cleared with protein-G agarose/Salmon Sperm DNA for 1 h at 4 °C 5% of material was used as input, and the remaining material was divided for incubation overnight at 4 °C with 30 μg of rabbit polyclonal anti-trimethyl H3K27 or with 30 μg of rabbit IgG as a control. The protein-G agarose/Salmon Sperm DNA was added for 1 h, beads were collected by centrifugation and sequentially washed for 3 min in 1 ml of low salt wash buffer (0.1% SDS, 1% Triton-X100, 2 mM EDTA, 20 mM Tris-HCl, pH 8.1, 150 mM NaCl), high salt wash buffer (0.1% SDS, 1% TritonX-100, 2 mM EDTA, 20 mM Tris-HCl, pH 8.1, 500 mM NaCl), LiCl wash buffer (0.25 M LiCl, 1% NP40, 1% deoxycholate, 1 mM EDTA, 10 mM Tris-HCl pH 8.1) and then twice with TE (10 mM Tris-HCl pH, 8.0, 1 mM EDTA). Beads were incubated two times for 15 min in 250 μl of the elution buffer (1% SDS, 0.1 M NaHCO3) at room temperature, supernatants were combined and incubated at 65 °C for 16 h in the presence of 0.2 M NaCl to reverse cross-link, and treated with proteinase K for 2 h at 45 °C. DNA was purified by p henol/chloroform extraction and precipitated with ethanol. DNA was re-suspended in 500 μl of the TE buffer. 5% of material was removed and used as second input in the analysis by real time PCR. 20 μg of salmon sperm DNA was added to the remaining material. Samples were boiled for 5 min and then kept on ice for 2 min. 50 μl of the 10× adjusting buffer (110 mM sodium phosphate, pH 7.0, 1.52 M NaCl, 0.55% Triton X100) was added to samples. Samples were incubated at room temperature for 20 min with 15 μg of anti-brdU antibody (BD Biosciences), followed by 20 min incubation with 35 μg of rabbit anti-mouse IgG. Samples were centrifuged at 18,000 g for 20 min and pellet was washed with the adjusting buffer and incubated for 2 h at 45 °C in the lysis buffer (50 mM Tris-HCl, pH 8.0, 10 mM EDTA, 0.5% SDS, 25 mg/ml proteinase K). DNA was purified by phenol/chloroform, precipitated with ethanol and used for analysis. All PCR reactions were performed with an Applied Biosystems StepOne Real-Time PCR system.

Block of DNA replication: To block DNA replication, cells were labeled with EdU for 30 min, induced to specific lineage for 24 hr in the presence of 2 mM thymidine. To restore DNA replication, thymidine was removed by washing, and cells were grown in culture media without thymidine for 4 hr, fixed, and analyzed by CAA with antibodies against biotin (EdU-labeled DNA) and corresponding TFs. The efficiency of the thymidine block of DNA replication was tested by labeling cells with 30 μM BrdU for 20 min in thymidine and after removal of thymidine, fixed with 4% formaldehyde, denatured in 2N hydrochloric acid for 20 min, washed in PBS and incubated with monoclonal anti-BrdU antibody (Exbio) followed by incubation with anti-mouse Alexa Fluor 488.

Gene expression analysis by RT-qPCR: Total RNA was isolated directly from freshly collected hESCs with TRIzol and for mESCs with High Pure RNA Isolation Kit. cDNA was synthesized by using 1 μg total RNA in a 20 μL reaction with Superscript III and oligo (dT) for hESC or random hexamers (Life Tech) for mECS. After reverse transcription was complete, one microliter of RNase H was added to each reaction tube, and the tubes were incubated for 20 min at 37 °C before proceeding to PCR. Real-time PCR was carried out on a 7500 Real-Time PCR System using the 2× Power SYBR green PCR master mix. GAPDH was used as an internal control. All PCR products were checked by running an agarose gel for the first time and by doing dissociation assay every time to exclude the possibility of multiple products. PCR analyses were conducted in triplicate for each sample.

Dilutions of antibodies used: For PLA: rabbit anti SIP1 (1:200), rabbit anti LMX1A (1:200), goat anti FOXA2 (1:200), rabbit anti-RAR beta (1:200), rabbit anti-HOXA5 (1:200), rabbit anti-HOXA3 (1:100), rabbit anti-H3K27me3 (1:2500), mouse anti-biotin for hESCs (1:1000), goat anti-biotin for mESC (1:1000), rabbit anti-UTX (1:400), rabbit ani-JMJD3 (1:400), rabbit anti-EZH2 (1:400), mouse anti-BrdU (1:100).

The experimental examples of the current invention are now described.

### Example 1: Assaying the Fate of Chromatin-Associated Proteins Following DNA Replication

New approaches to study the fate of chromatic-associated proteins following DNA replication were developed. In particular, the Chromatin Assembly Assay (CAA) examines the assembly of chromosomal proteins on nascent DNA at a single-cell level (FIG. 1A). In this assay, DNA is labeled in living cells in a pulse-chase manner with EdU, which is then chemically conjugated with biotin using a ‘Click-iT’ reaction. The proximity of a protein of interest to nascent DNA is then examined by the Proximity Ligation Assay (PLA, Olink, Bioscience) using antibodies to biotin and the protein of interest. PLA is a powerful technique that detects single molecule interactions. Cells can be then examined for specificity of interactions (e.g. specific PLA signals are detected only in EdU-labeled nuclei), and by assessing tissue specificity using relevant protein markers. Reliable pulse-chase EdU labeling in cells can be detected from 3-5 min to several hours. A gene-specific re-ChIP assay with BrdU-labeled DNA, previously used in Drosophila and in human HPCs (FIG. 1B), complements this CAA approach. In this assay, DNA is pulse-labeled with BrdU, chromatin is precipitated with the antibody of interest, and DNA is then denatured and precipitated with antibody against BrdU, followed by qPCR. This method allows the examination of the accumulation of proteins at specific binding sites over time after DNA replication. With both assays, a detailed picture of the behavior of proteins at their DNA binding sites at the single-cell level during and after DNA replication can be obtained.

### Example 2: Dynamics of Accumulation of H3K27me3 on Nascent DNA of Cytokine-Induced Multipotent HPCs, Pluripotent ESCs and Naïve T Cells

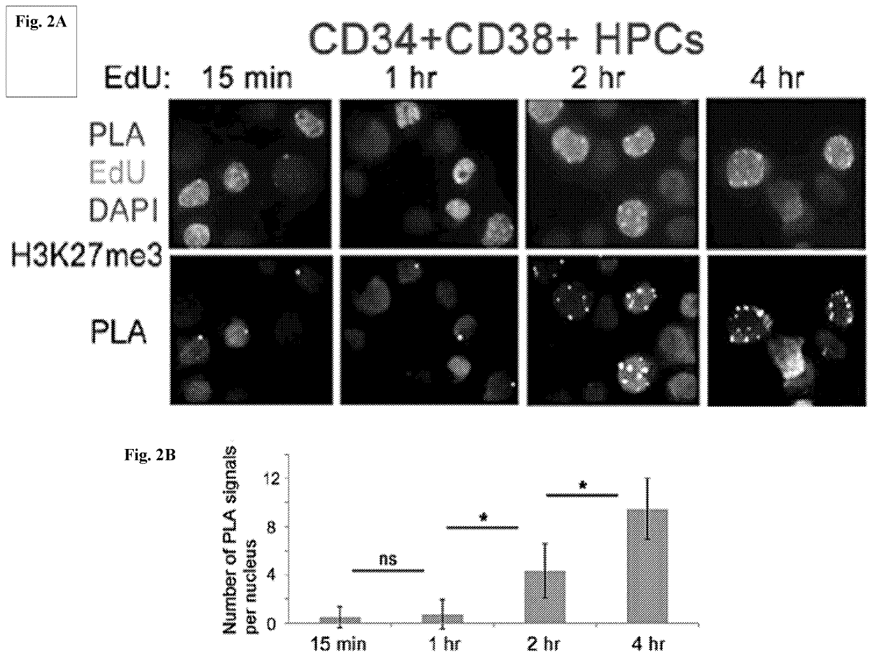

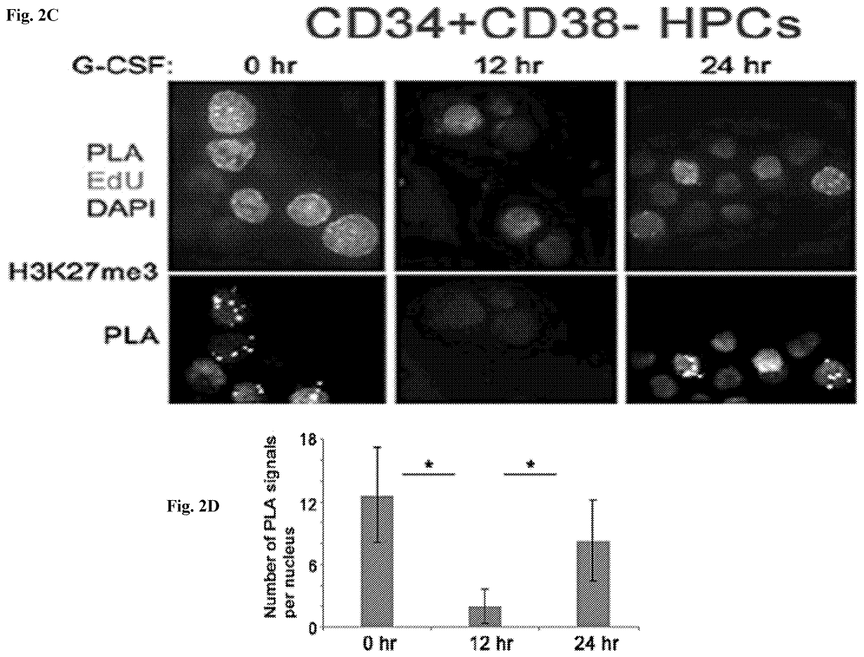

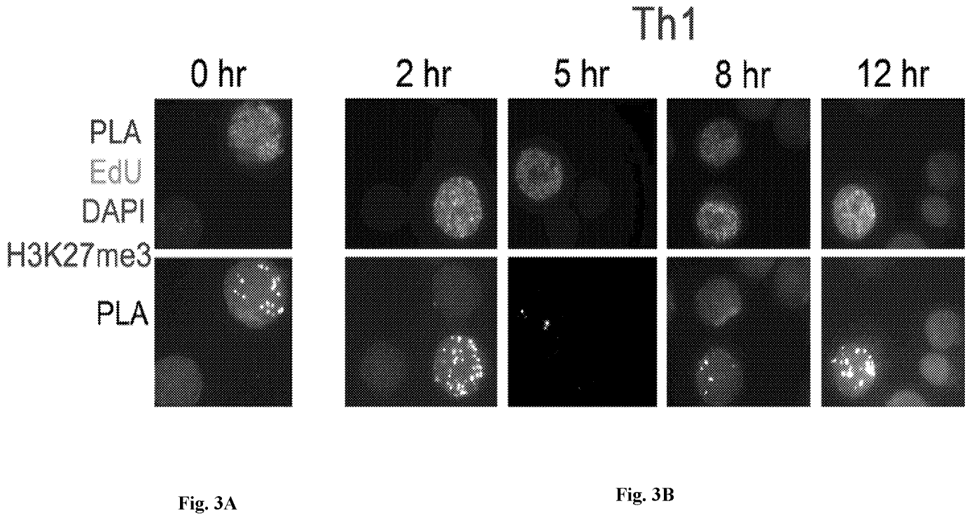

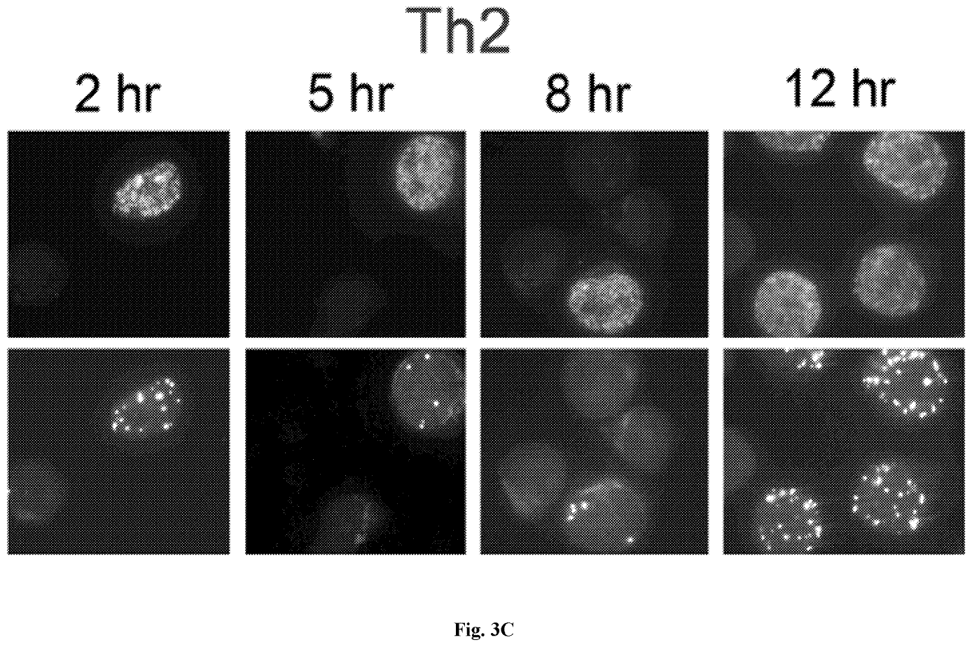

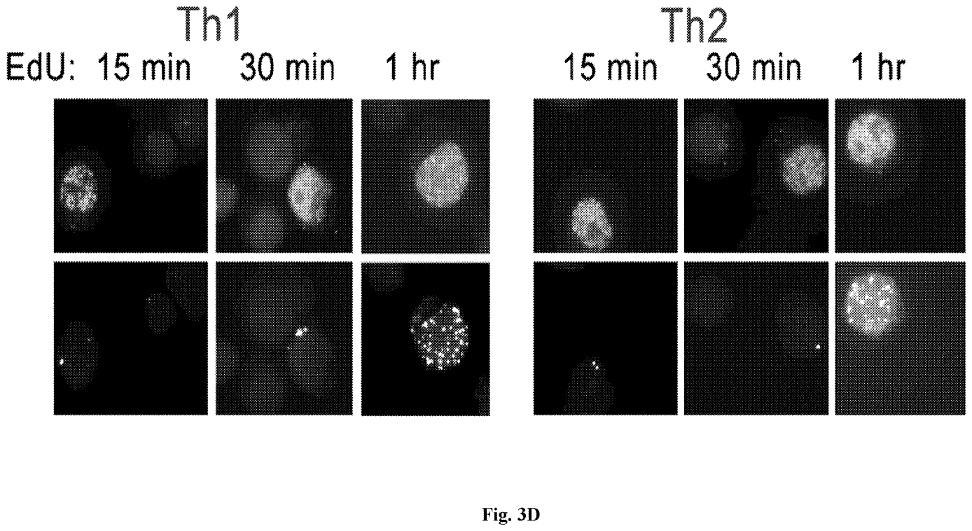

Using the CAA, it was observed that accumulation of post-replicative H3K27me3 on nascent DNA of normal CD34⁺CD38⁺ HPCs is significantly delayed (1-2 hr after DNA replication) (FIGS. 2A and 2B). By contrast, H3K27me3 accumulates very rapidly on nascent DNA of CD34⁺CD38⁻HPCs, and is becoming delayed after a 12-hr induction with G-CSF or M-CSF (FIGS. 2C and 2D). To examine whether this is a common phenomenon for other hematopoietic cells, differentiation of specialized naive T cells was assayed before and after priming by exposure to anti-CD3-TCR and anti-CD28 antibodies and exposure to polarizing cytokines to result in distinct T-cell subsets. Similar to HPCs, a delay in accumulation of H3K27me3 in nascent chromatin following differentiation of T cells into Th1 and Th2 lineages was detected (FIGS. 3A, 3B, and 3C). This delay was detected from 5 to 8 hr following induction to both lineages, and it lasted for over 30 min after DNA replication (FIG. 3D). Importantly, a similar delay was also detected during differentiation of human and mouse embryonic stem cells (ESCs) into neuronal lineages. Thus, irrespective of the cell type and differentiation status, accumulation of H3K27me3 after DNA replication undergoes a genome-wide temporal delay during very early stages of lineage determination.

### Example 3: Accumulation of H3k27me3 on Nascent DNA Blocks Recruitment of Key TFs to DNA During Normal Hematopoiesis

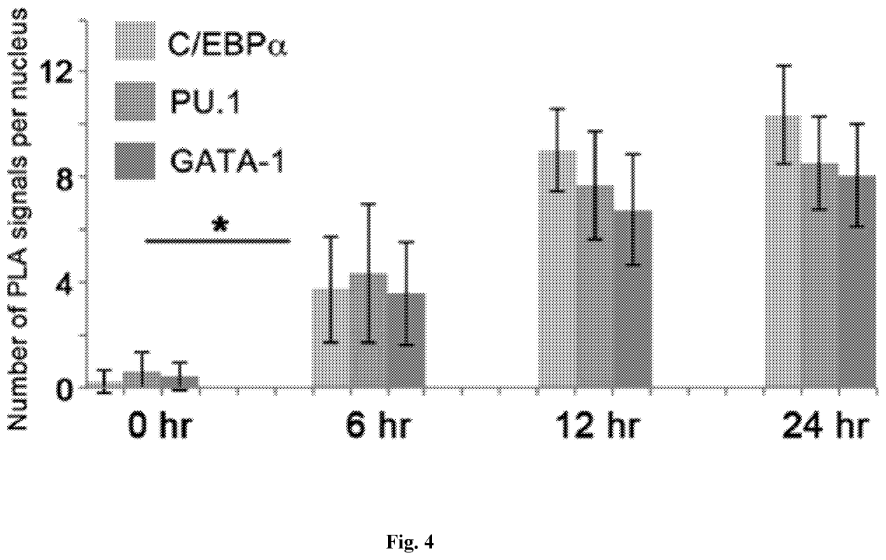

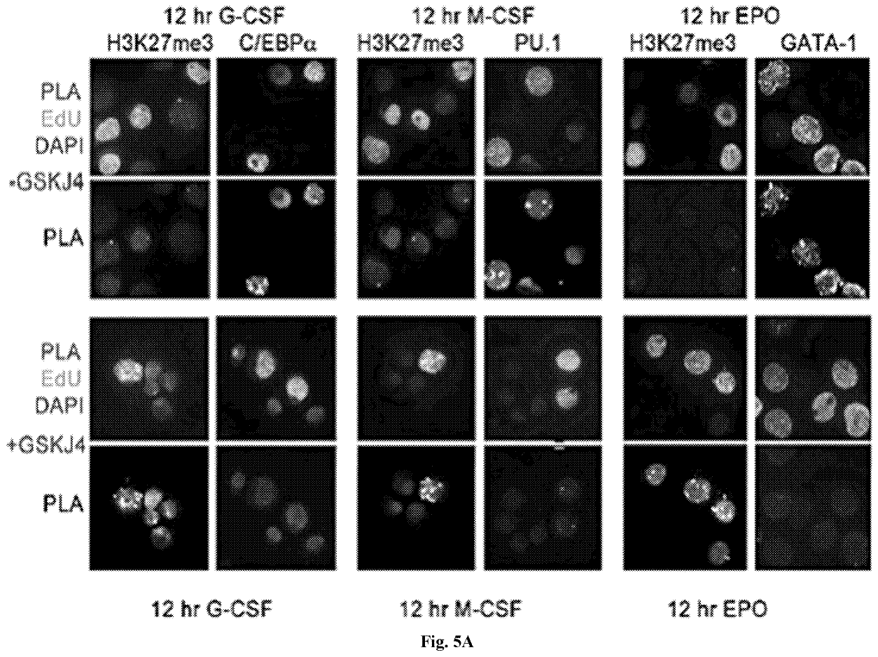

High content of nucleosomes with H3K27me3 correlates with high density of nucleosomes. The absence of H3K27me3 in nascent chromatin during early stages of differentiation may lead to a lower density of nucleosomes on newly synthesized DNA. Without wishing to be bound by theory, it was hypothesized that this chromatin structure facilitates the recruitment to DNA of lineage-specific TFs that are induced in response to differentiation stimuli. This hypothesis was examined experimentally by first testing the kinetics of association of lineage-determining TFs following induction of cell differentiation. During induction of differentiation of h/mESCs and HPCs, lineage-determining TFs were first detected on DNA at the time of delayed accumulation of H3K27me3 on nascent DNA. Three lineage-specific myeloid and erythroid TFs, C/EBPα, PU.1 and GATA-1 start to recruit to DNA unexpectedly early, 6 hr after induction of HPCs differentiation, at the time of de-condensed nascent chromatin (FIG. 4). Similar results were obtained with a number of TFs during neuronal differentiation of h/mESCs and for the lineage-specific TFs T-bet and GATA3 during differentiation of T cells into the Th1 and Th2 lineages.

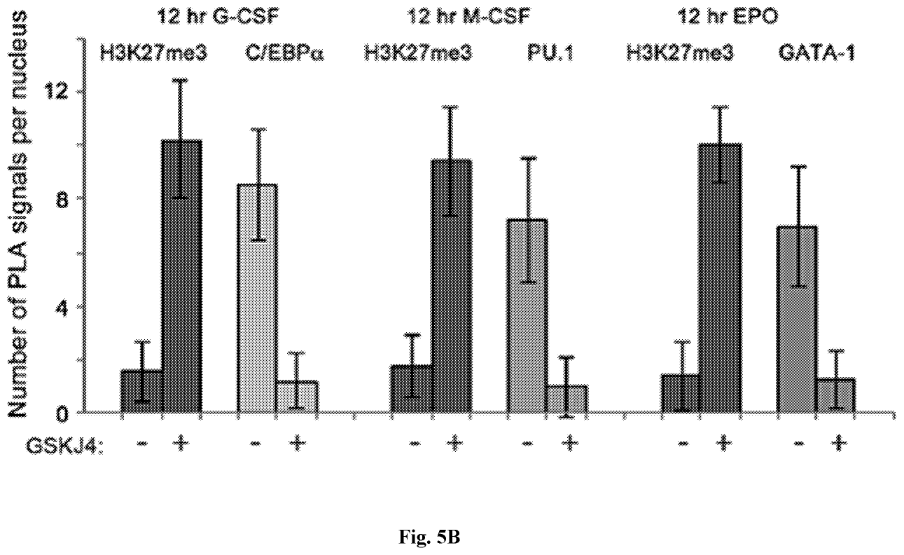

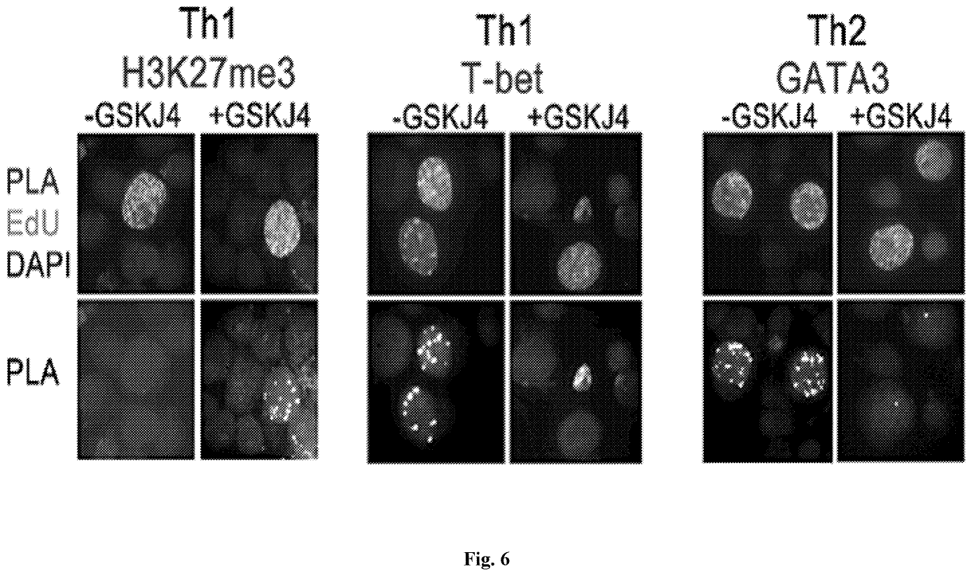

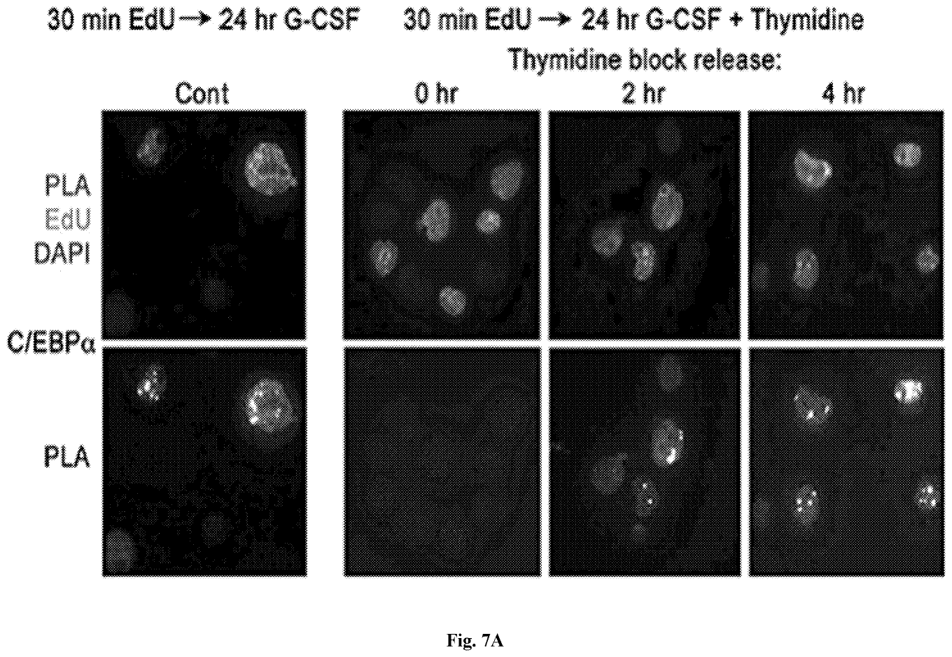

To further assess the importance of the structure of nascent chromatin, the de-condensation of nascent chromatin was inhibited, and the resulting inability of lineage-determining TFs to bind DNA was observed. Accumulation of H3K27me3 following DNA replication was strongly increased when CD34⁺ HPCs were exposed to GSKJ4 (FIG. 5A, bottom rows), the inhibitor of the H3K27me3 de-methylases (KDMs) UTX and JMJD3, thus signifying condensation of nucleosomes arrays in nascent chromatin. Treatment with GSKJ4 strongly inhibited recruitment of the TFs C/EBPα, PU.1 and GATA-1 (FIG. 5A, right columns). Results were quantified in FIG. 5B. Similarly, condensation of nascent chromatin following treatment of T cells with GSKJ4 led to inhibition of recruitment of T-bet and GATA3 to nascent DNA (FIG. 6). Without wishing to be bound by theory, these results suggest that chromatin that lacks H3K27me3, and therefore contains de-condensed arrays of nucleosomes after DNA replication, is essential for recruitment of fate-specifying TFs during very early stages of differentiation of ESCs, HPCs and T cells.

### Example 4: Recruitment of Lineage-Determining TFs Occurs Exclusively During Early Stages of DNA Replication

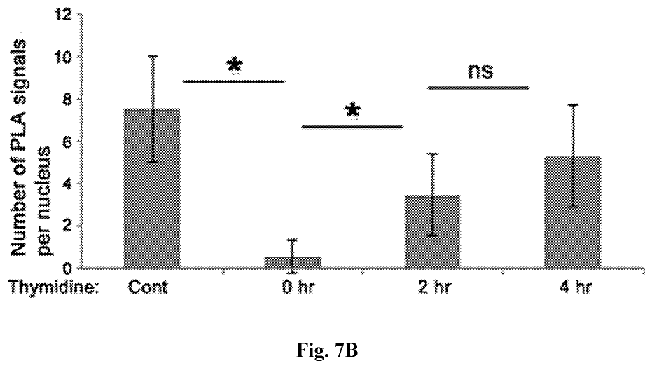

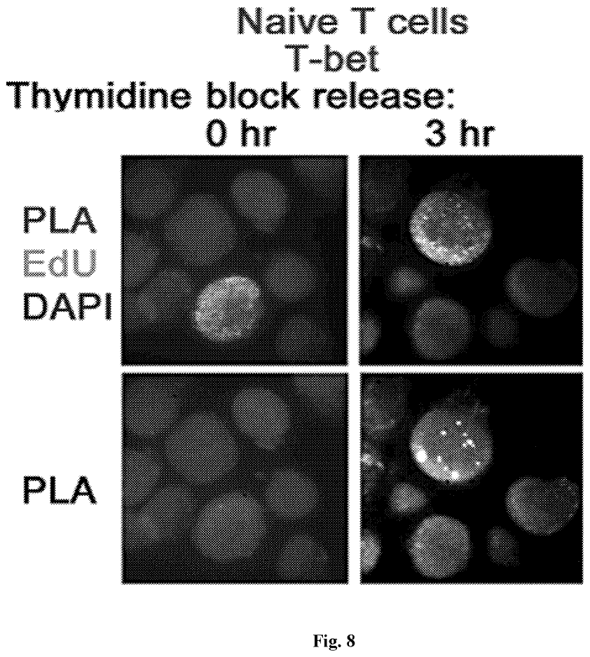

The results above suggested an examination of whether the recruitment of TFs to DNA occurs exclusively during the early stages of replication. Cytokine-treated hESCs or T cells induced to the Th1 lineage were labeled with EdU for 30 min, and grown in the presence of thymidine to prevent S phase entry. Such treatment efficiently blocked DNA replication, and prevented association of C/EBPα in HPCs (FIG. 7A). Re-initiation of DNA synthesis 3-4 hr after release from thymidine block leads to efficient recruitment of this TF to DNA (FIG. 7B). Similarly, blocking S phase entry by thymidine completely inhibited recruitment of T-bet to DNA in T cells, and release of thymidine block led to fast accumulation of T-bet on labeled DNA (FIG. 8). Without wishing to be bound by theory, these results suggest that recruitment of TFs following induction of differentiation of CD34⁺ HPCs and T cells occurs exclusively during early periods after DNA replication.

### Example 5: Association of the KDM UTX with Nascent DNA is Increased Following Induction of Cell Differentiation

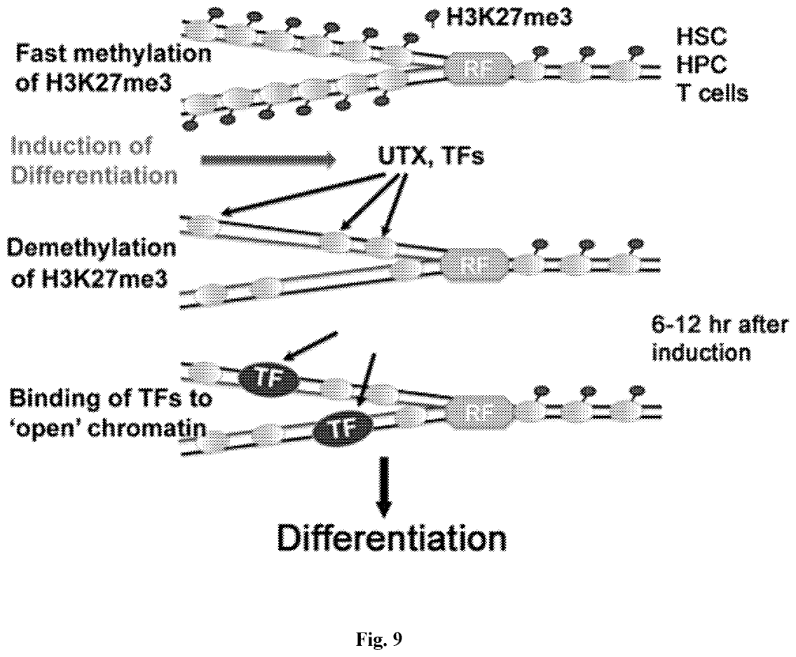

Changes in the rate of accumulation of H3K27me3 on nascent DNA may depend on the relative activities and/or amounts of antagonistic enzymes, H3K27 HMTs and KDMs. It was observed that the major H3K27me3 HMT EZH2 is present on DNA before and after induction of h/mESCs, HPCs and T cells. However, the amount of the major H3K27me3 KDM UTX associated with DNA is very low in undifferentiated cells, and it is significantly increased following induction of differentiation of these cells. Without wishing to be bound by theory, it is likely that a strong increase in the amount of chromatin-associated UTX masks the H3K27me3 activity of EZH2 leading to de-methylation of H3K27me3 on nascent DNA during the first several hours after induction of cell differentiation. Importantly, inhibition of UTX activity not only blocks binding of TFs (FIGS. 5 and 6), but also strongly inhibits differentiation of ESCs, HPCs and T cells. Together, these results suggest that irrespective of cell type and lineage specification, transient, low density chromatin just after DNA replication is essential for recruitment of lineage-specifying TFs to DNA and, as a result, for cell differentiation. The new molecular mechanism of induction of differentiation in normal cells is shown in FIG. 9.

### Example 6: TCL Cells Lost the Ability to De-Condense Nascent Chromatin

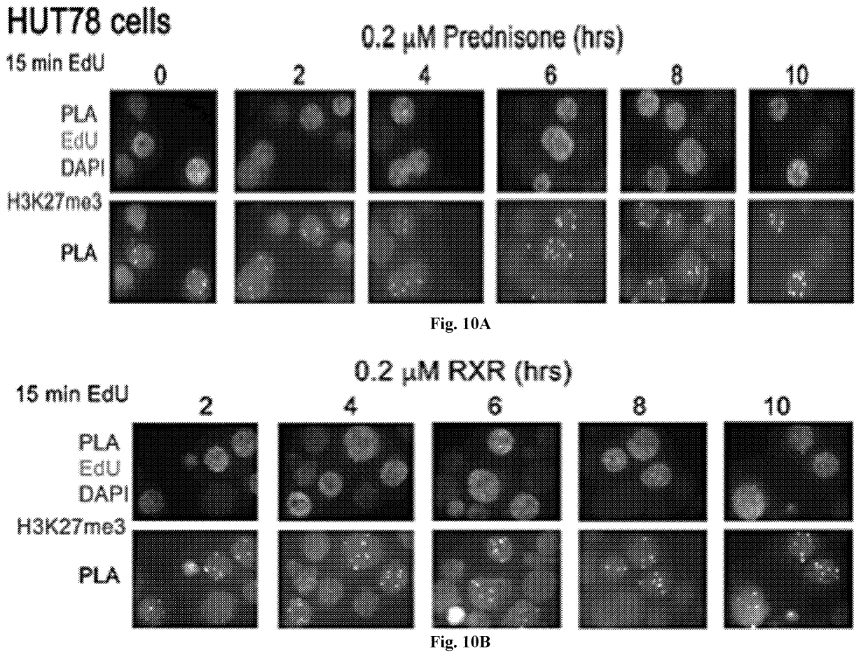

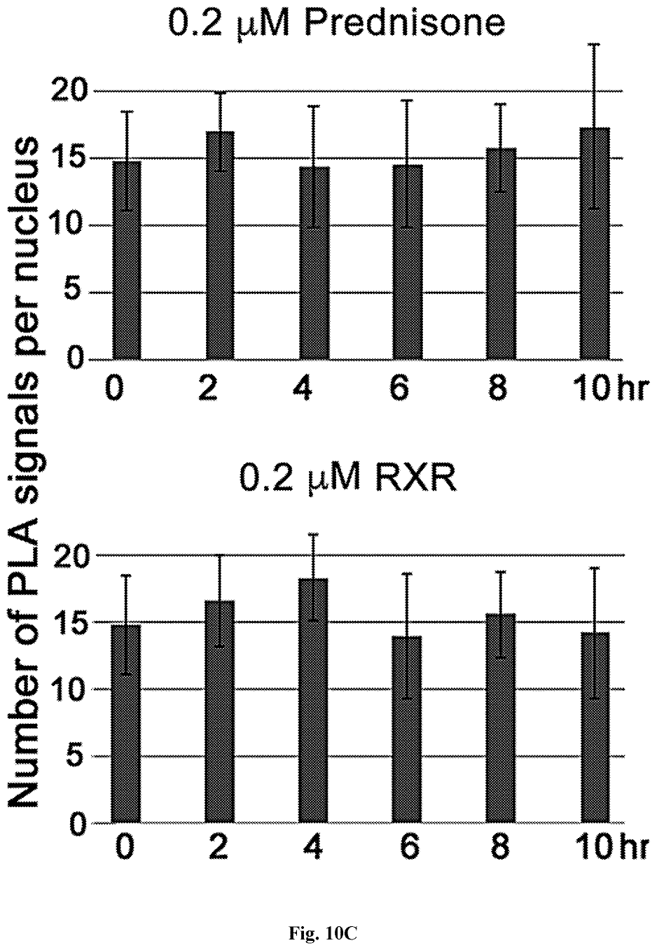

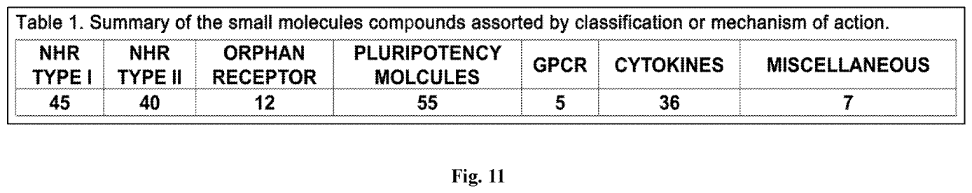

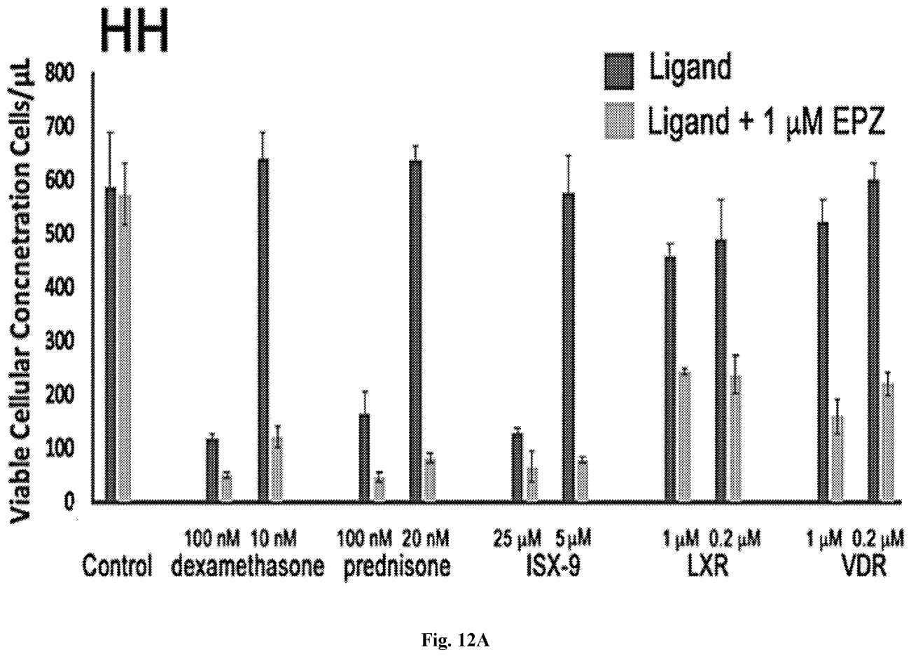

Since TCL cells have lost the ability to differentiate into mature lineages, or display features of aberrant cell maturation, it was next assessed whether the mechanism that allows normal cells to de-condense nascent chromatin is lost or altered in leukemic cells. To test this possibility, nascent chromatin structure of several types of TCLs, including SS lines HUT78 and HH, T-ALL line Jurkat E6.1, and blast cells from a patient with T-PLL (CD3⁺CD4⁻CD8⁺) was assessed. In all these cells, high accumulation of H3K27me3 at 15 min after DNA replication during extended periods of culturing (HuT78 cells in FIG. 10A) was observed. Next, it was determined whether nascent chromatin in TCLs may potentially de-condense following induction with NR ligands prednisone and RXR that, in high concentrations, exert growth suppressive effects on leukemic cells, including T cell leukemia. The high level of H3K27me3 on 15 min-labeled nascent DNA remained unchanged through 10 hr of induction of HUT 78 cells with both NR ligands (FIGS. 10A and 10B, quantified in FIG. 10C). Without wishing to be bound by theory, this suggested that, in contrast to cytokine-induced differentiation of normal T cells (FIG. 3), de-condensation of nascent chromatin does not occur following NR ligand treatment of TCL cells (FIG. 10).

### Example 7: Inhibition of Viability of TCL Cells by TF-Activating Small Molecule Ligands is Strongly Enhanced by De-Condensing Nascent Chromatin

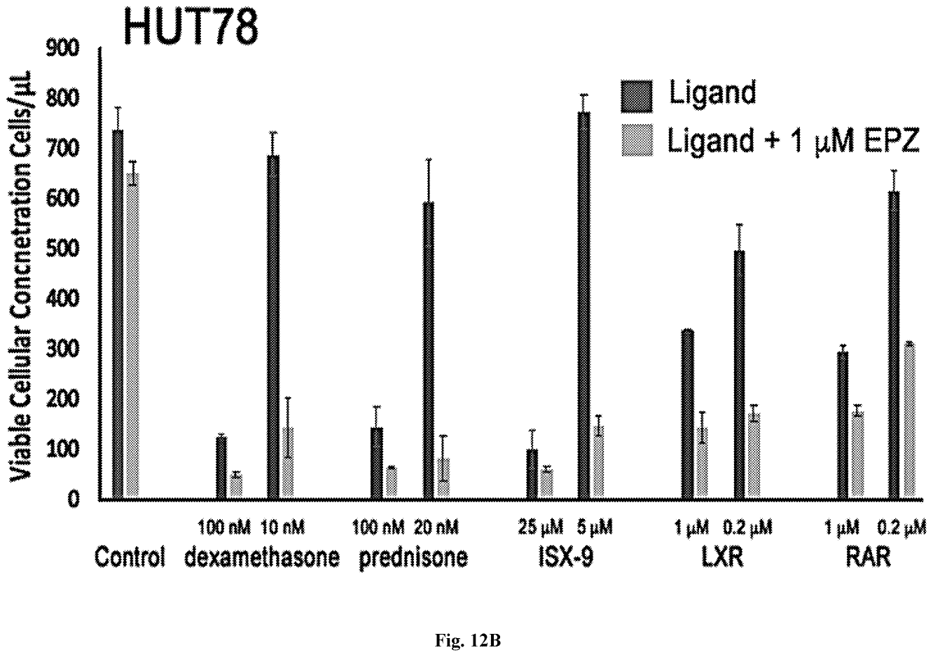

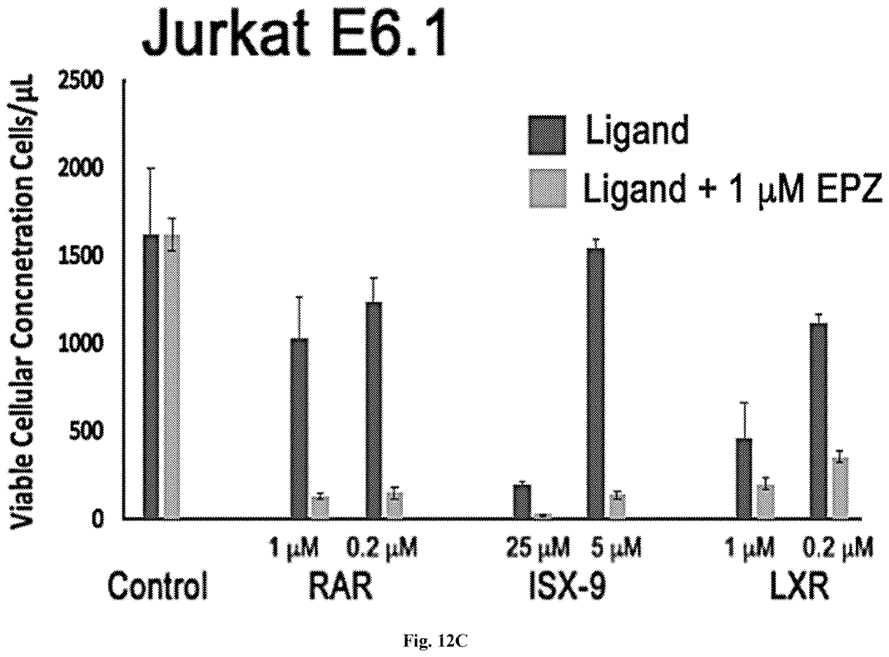

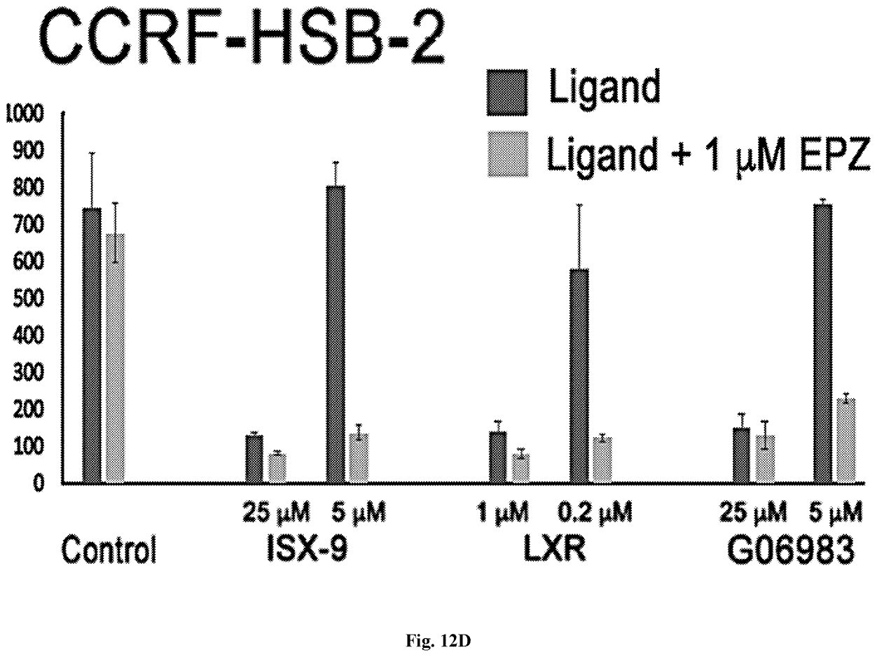

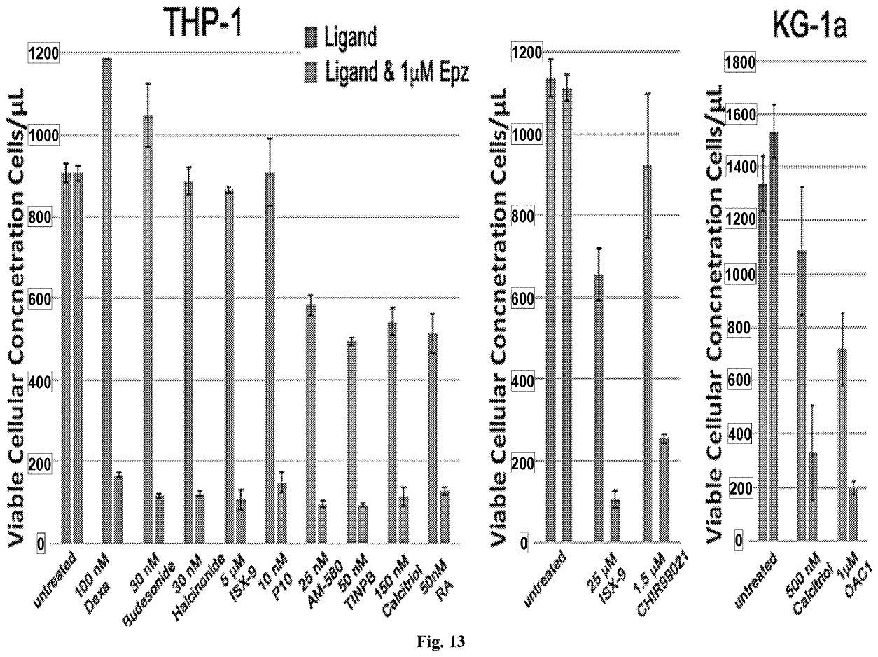

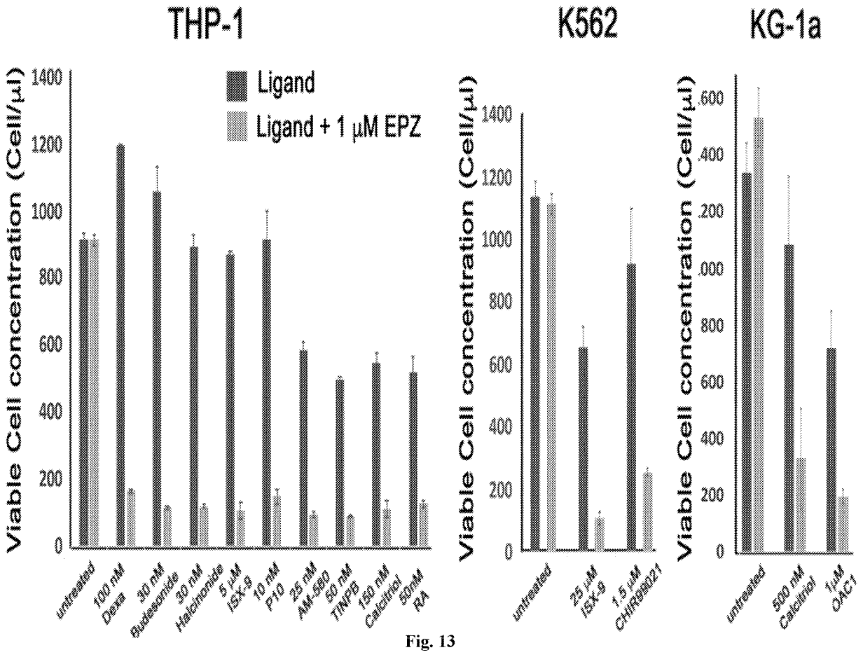

To test the idea that restoring the ability of cells to de-condense nascent chromatin may enable reprogramming of TCLs, a two-step induction was devised: Cells were first treated with the inhibitors of the EZH1/2 HMTs, and then induced with small molecule ligands known to activate specific TFs. Two inhibitors of EZH1/2, GSK126 and Tazemetostat (EPZ-6438, herein called EPZ), were used. Both inhibitors induced a marked decrease in the levels of global and nascent chromatin marked with H3K27me3 when used for 24 hr in the 0.5-1 μM range (tested by CAA, see FIG. 13B). Since different types of TCL cells may not possess receptors for TF-activating small molecule ligands because of their differentiation or mutation status, a screen was setup that would allow examining ligands for multiple different families of receptors. A library of small molecules was compiled that included ligands for nuclear hormone receptors, cytokines, ligands for GPCRs, and small molecules that are used to reprogram mature cells into pluripotent cells (FIG. 11). Since different ligands may induce reprogramming of leukemic cells into different lineages, it was not possible to use specific differentiation markers at initial stages of these screens, and therefore cell viability was used as a readout for the initial screens. The earliest effects on cell viability were observed after 5 days of treatment, and therefore screens for cell viability (flow cytometry with Sytox green) were performed at days 8-11. Screens of the SS cells HUT78, HH and of the T-ALL cells Jurkat E6.1 and CCRF-HSB-2 cells produced several important results; the results for some efficient effector molecules are shown in FIGS. 12A-12D. Namely that in line with previous studies, SS HH and HUT78 cell lines were sensitive to steroids, while the T-ALL lines Jurkat E6.1 and CCRF-HSB-2 were not. The effects of the LXR, VDR and RAR nuclear hormone receptors were also uncovered on some of these cells. Further, while the results in HH and HUT78 lines were in general similar, there were clear differences. This emphasizes the need for screening particular subtypes of leukemic cells with a large number of ligand/effector molecules to identify best inducer. Most importantly, effective ligand molecules shown in FIG. 12 exhibited greatly enhanced growth suppression when they were used after inducing chromatin de-condensation with EZH1/2 inhibitors. In addition to detecting synergism between EZH inhibitors and newly discovered small molecule effectors, these results reveal that the efficient concentrations of known inducers can be greatly diminished. For example, effective concentrations of steroids can be reduced 5-10 fold in the presence of the EZH inhibitors.

### Example 8: Inhibition of Viability of AML Cells by TF-Activating Small Molecule Ligands is Strongly Enhanced by De-Condensing Nascent Chromatin

Similar experiments were performed with the AML cell lines THP-1, K562 and KG-1a. The results for some efficient small molecule inducers are shown in FIG. 13. ISX-9 induced inhibition of cell viability in all AML cell lines (FIG. 13).

### Example 9: Inhibition of Viability of B-ALL Cells by TF-Activating Small Molecule Ligands is Strongly Enhanced by De-Condensing Nascent Chromatin

Similar experiments were performed with the B-ALL cell lines SEM, SUP-B15, RS4;11, 697, and BV173. The results for some efficient small molecule inducers are summarized in FIG. 11 and FIG. 14. Some small molecules induced inhibition of cell viability in two or more B-ALL cell lines (FIG. 14).

### Example 10: De-Condensed Nascent Chromatin is Essential for Binding of the Ligand-Activated TFs to DNA

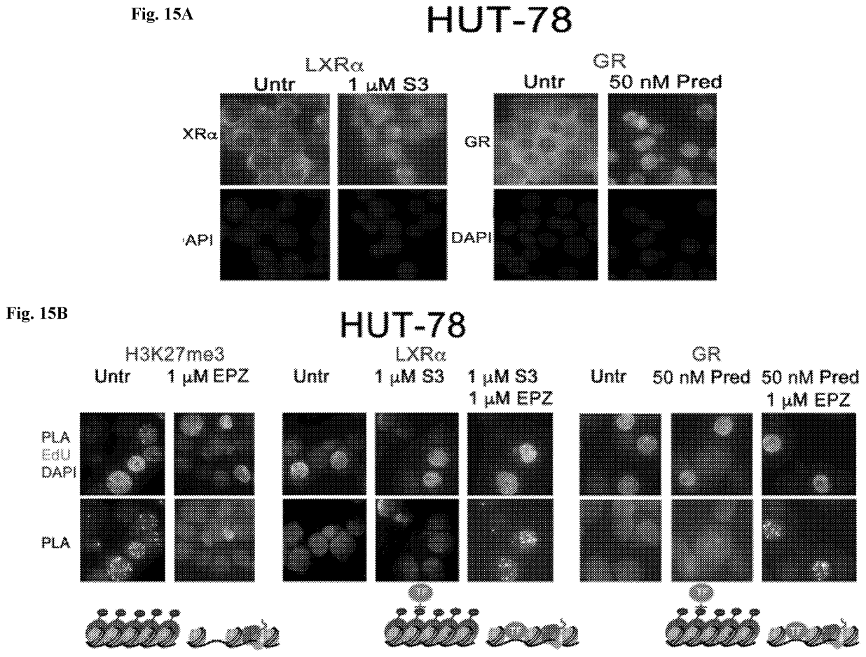

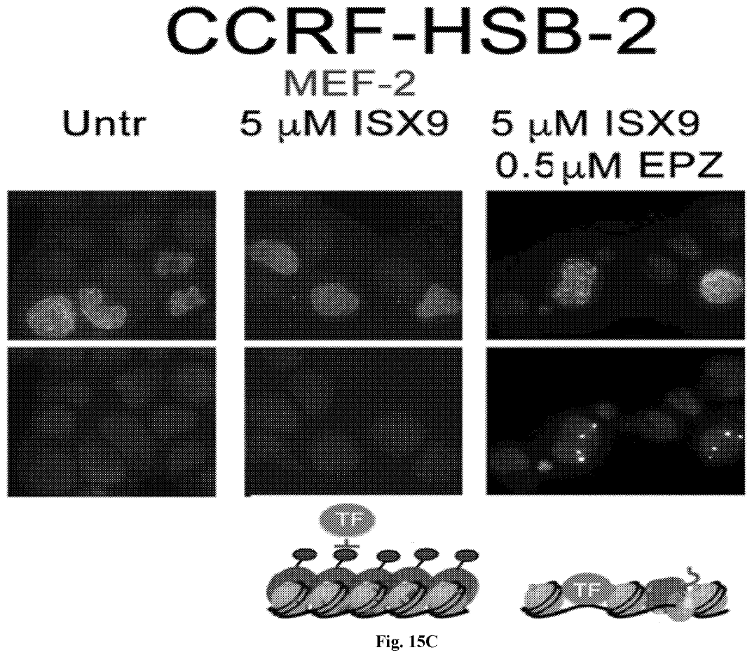

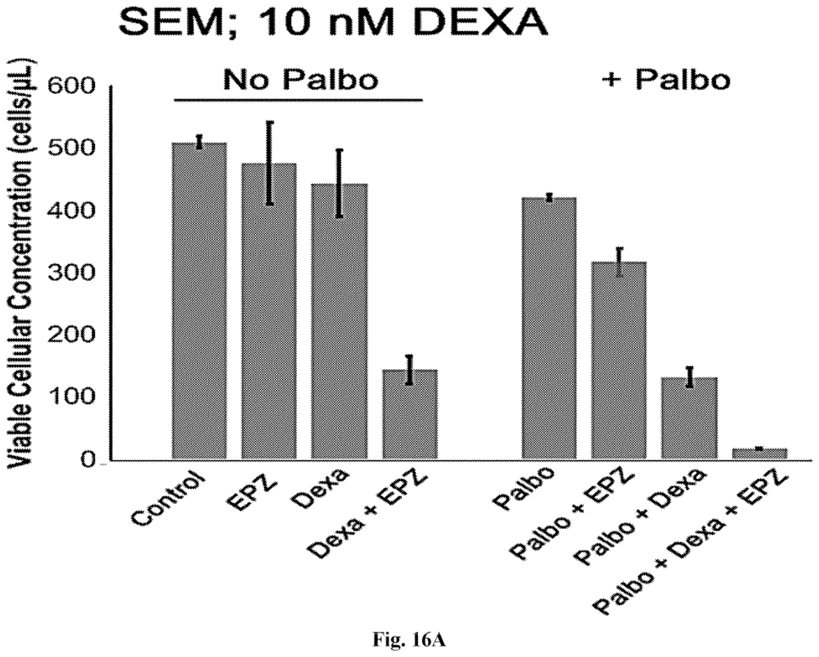

To understand whether binding of the ligand-activated TFs to DNA is indeed dependent on the de-condensed state of nascent chromatin, the liver X receptor (LXRα) and glucocorticoid receptor (GR) were chosen, the ligands of which showed strong synergistic effects with the EZH1/2 inhibitors on cell viability of HH and HUT78 cells (FIG. 12A,B). In untreated HUT78 cells, LXRα and GR were detected mostly in the cytoplasm. Following induction with GW3965 and prednisone (ligands for LXRα and GR, respectively), these ligand-activated receptors became exclusively nuclear, which is consistent with their mode of functioning as type I nuclear hormone receptors (FIG. 15A). Treatment with 1 μM EPZ for 24 hr completely inhibited accumulation of H3K27me3 on nascent chromatin, suggesting its de-condensed state (FIG. 15B, left). As expected, un-liganded LXRα and GR do not bind to nascent DNA in untreated HUT78 cells. Importantly, even at high concentrations of ligands, they are still not able to bind to DNA following translocation to the nucleus. However, pre-treatment of cells with EPZ leads to very strong binding of both NRs to DNA (FIG. 15B, right). In Example 7, the small molecule isoxazole (ISX9), was effective in inhibiting cell viability of all tested TCL cells (FIGS. 12A-12D). ISX9 triggers robust neuronal differentiation of adult neural stem cells and reprograms mouse fibroblasts into neurons. ISX9 upregulates multiple neuron-specific genes, including the neural-fate mastering genes NeuroD1, Ngn2 and MEF2. In case of MEF2, ISX9-triggered Ca2⁺ influx freed this TF from repression by HDAC5 through chaperoned nuclear export of phosphorylated HDAC. This analysis confirmed that activated MEF2 binds to nascent chromatin. However, this binding occurs only following de-condensation of chromatin by EPZ (FIG. 15C). These results suggested that despite activation with the ligand/effectors, these TFs cannot bind to DNA with condensed nascent chromatin. Thus, de-condensing nascent chromatin is an essential step in binding of ligand-activated TFs to DNA.

### Example 11: Synchronizing Cells in the Beginning of the S-Phase by the Cdk4/6 Inhibitor Palbociclib Enhances the Efficiency of Combination Treatment with EZH1/2 Inhibitor Tazemetostat (EPZ-6438) (EPZ) and Dexamethasone or ISX-9 on Cell Survival of the B Cell Leukemic ALL Cell Line SEM

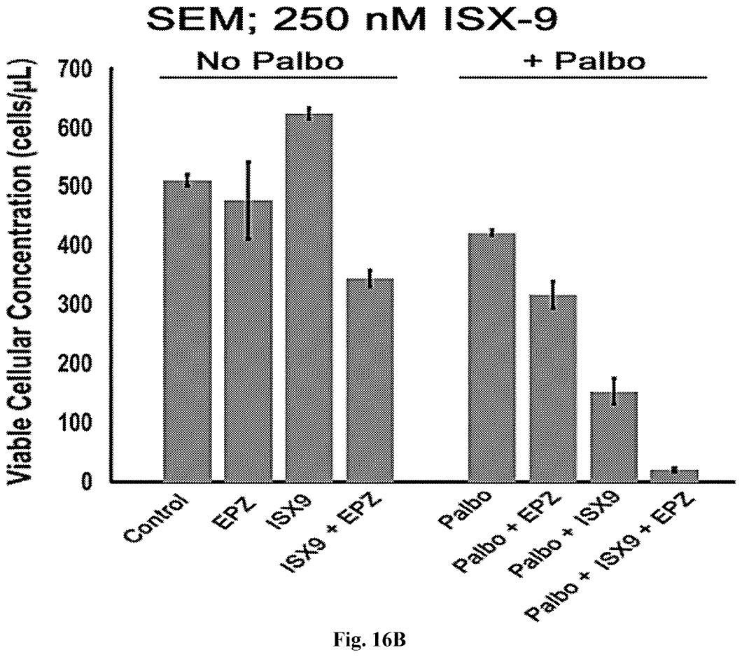

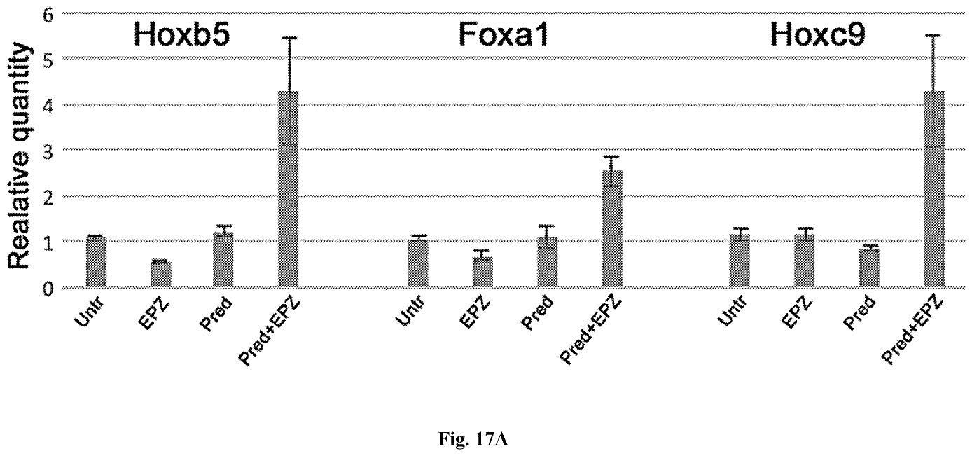

To understand whether synchronizing leukemic cells further enhances the efficiency of inhibiting cell viability, the B-ALL SEM cells were pre-treated 500 nM of the Cdk4/6 inhibitor Palbociclib, and then were released into the beginning of the S-phase by washing of this inhibitor. This pre-treatment was followed by treatments with 100 nM EPZ, followed by treatments with 10 nM Dexamethasone (FIG. 16A, DEXA) or 250 nM ISX-9 (FIG. 16B). Without wishing to be bound by theory, these results suggest that synchronizing SEM cells strongly increases the effects of both activating molecules on inhibition of survival of SEM cells.

### Example 12: Combination EZH1/2 Inhibitor EPZ and Prednisone Treatment Enhances the Expression of Differentiation-Associated Genes

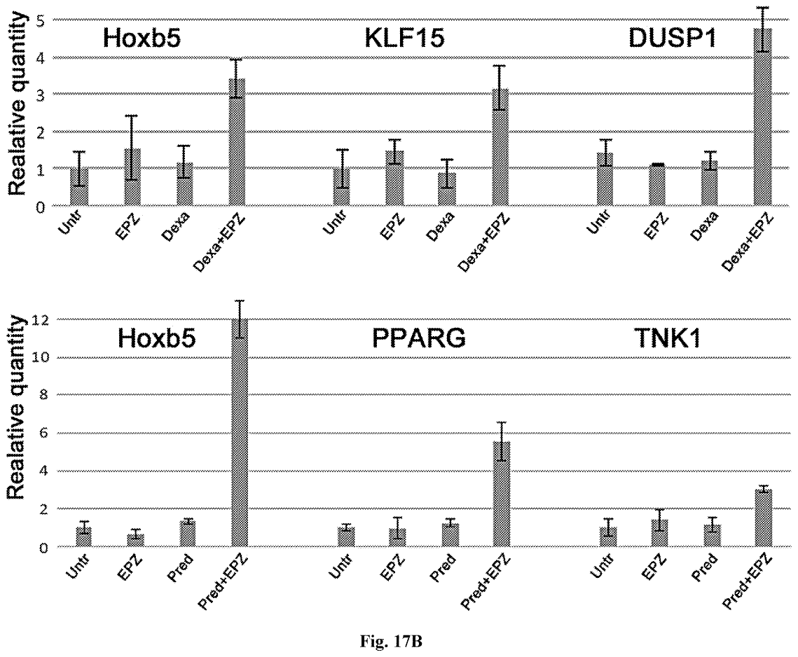

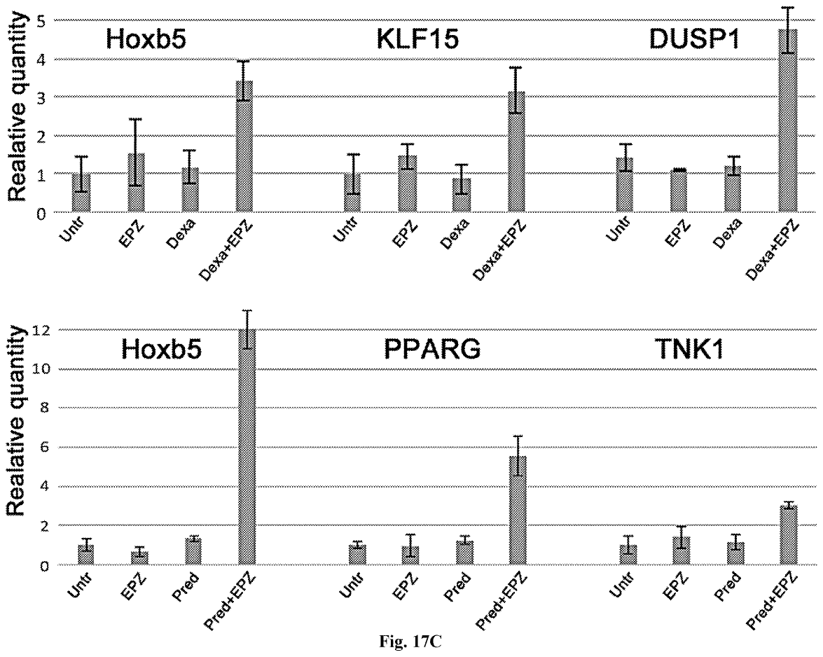

Cultured cells of the AML line THP-1 were untreated (Untr) or treated with 1 mM EPZ for 24 hr, or with 1 mM of Prednisone (Pred) for 24 hr, or with the combination of EPZ and Pred (FIG. 17A). Graphs show induction of transcription of GR target genes. (FIG. 17B) SEM cells, a B acute lymphoblastic leukemia cell line, were untreated (Untr) or treated with 1 mM EPZ for 24 hr, or with 20 nM of Dexamethasone (Dexa, top graph), or 100 nM of Prednisone (Pred, bottom graph) for 24 hr, or with the combinations of EPZ and Dexa or Pred. Graphs show induction of transcription of GR target genes. (FIG. 17C) Cells of the T-ALL cell line HH were untreated (Untr) or treated with 1 mM EPZ for 24 hr. Top graph, cells were then treated with 50 nM of Prednisone (Pred) for 24 hr or with the combinations of EPZ and Pred. Bottom graph, cells were then treated with 1 mM of ISX-9 for 72 hr or with combinations of EPZ and ISX9. Analysis by qRT-PCR. In each cell line, expression of differentiation-associated genes was significantly upregulated by the combination of EPZ and Dexamethasone or Prednisone, while the use of each agent alone had no statistical difference from untreated cells. These results demonstrate that the combination of EZH1/2 inhibition with Dexamethasone or Prednisone treatment could be an effective strategy for inducing differentiation in poorly-differentiated lymphoma tumors cells.

### Example 13: A Personalized Therapy Model of EZH1/2 Inhibition in B-ALL

Cancer, especially leukemias and lymphomas, can result from a wide variety of genetic mutations and abnormalities in gene expression, even between individual patients diagnosed with the same cancer type. These individual and unique differences and may contribute to the varied responses to common cancer treatments observed in the clinic. Recent advances in rapid phenotyping, genotyping, and functional characterization of tumor specimens has led to the development of personalized or precision medicine in cancer treatment. Here, tumor specimens from individual patients can be screened ex vivo with various treatment combinations in order to determine which would be the optimal treatment. This optimization would be a key part of combination therapies involving histone modification and reprogramming (i.e. differentiation induction), as particular receptors/TFs in each cancer can be mutated, and therefore not functional. Identifying the inducer molecules that are most efficacious for a particular cancer patient would ensure a more successful treatment outcome.

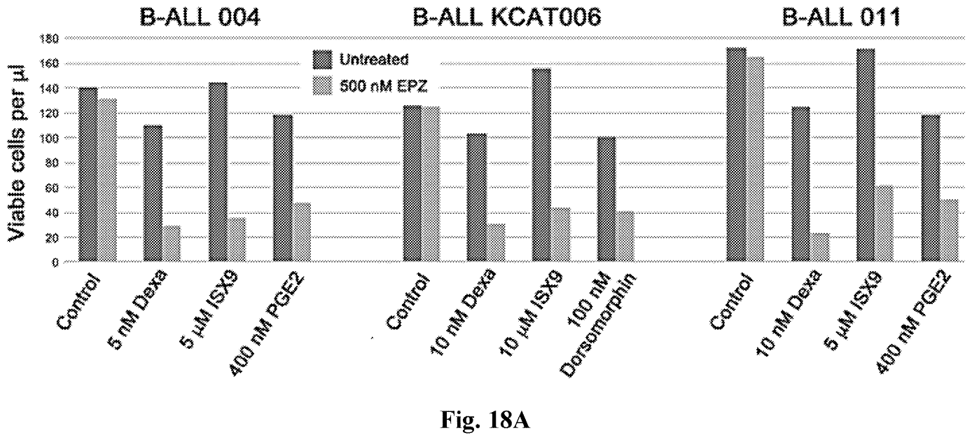

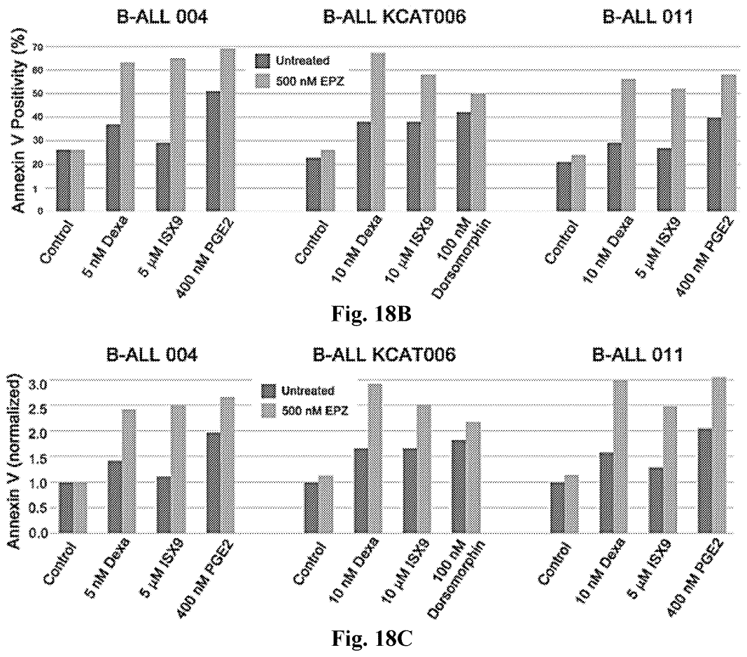

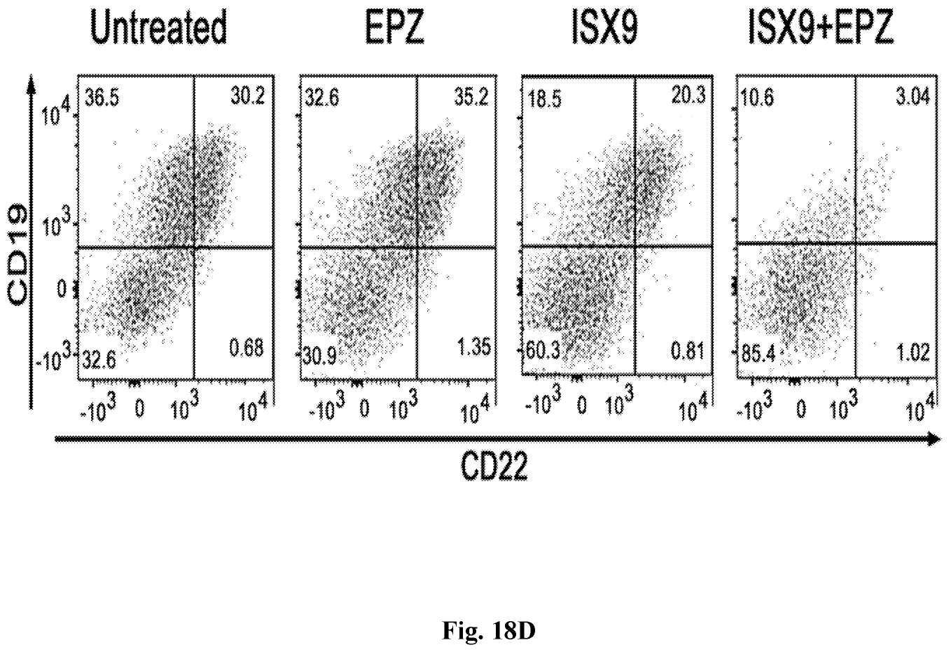

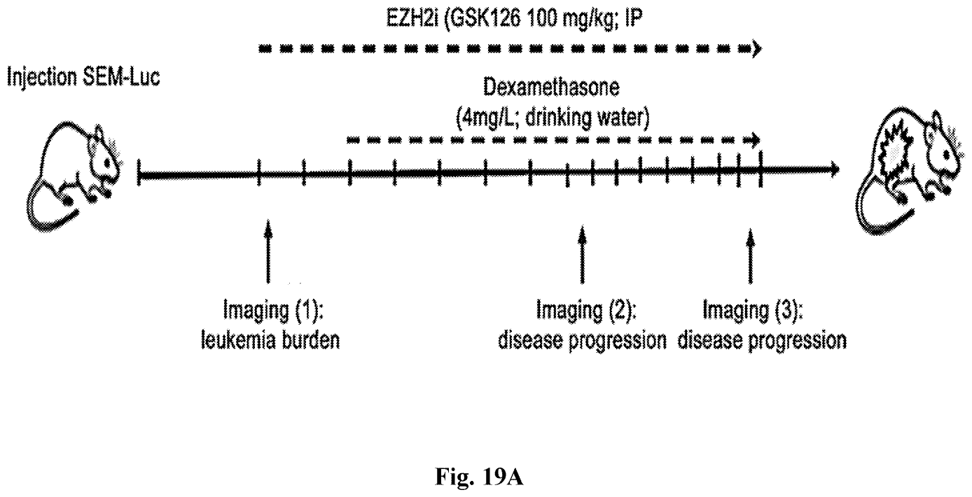

A series of studies was undertaken in order to observe the effects of combination EZH1/2 inhibition with several reprogramming agents (e.g. differentiation-inducing compounds) in cells derived from B-ALL patients. Samples from three patients were cultured in vitro until a minimum of 10% of cells were in S-phase, indicating actively replicating populations. Patient samples were then plated and treated as shown previously for SEM with or without 500 nM EPZ for 24 hours followed by treatments of Dexa, ISX9, PGE2, or Dorsomorphin with the given concentrations. All results shown are for 7 days after addition of inducers. All patient samples were cultured in Stem Cell Factor Media with a cocktail of cytokines: IL-3 (10 ng/mL), IL-6 (10 ng/mL), IL-7 (10 ng/mL), FLT-3 (20 ng/mL), Stem Cell Factor (20 ng/mL). FIG. 18A illustrates viable cell concentration per mL using Sytox Green via flow cytometry. FIG. 18B illustrates Annexin V positivity via flow cytometry. FIG. 18C illustrates the same data as a normalized quantity to untreated control group. FIG. 18D illustrates cells from B-ALL patient 004 grown and treated as described in FIGS. 18A-C. Immunophenotyping via flowcytometry for the cell surface antigens CD19 and CD22. Each cell line demonstrated dramatically increased Annexin V positivity and decreased viability when treated with the combination of EPZ with Dexa, ISX9, or PGE2, while treatment with EPZ alone had little effect compared to untreated controls.

Together, these data show that the combination treatment strategy of the present invention can be applied in a ‘personalized manner’. Leukemic cells from patients can be established in short-term culture for about 10 days. During this time, these cells are screened for the best possible reprogramming agent, which causes reprogramming (e.g. differentiation), loss of cell viability and apoptosis. These studies screen about 5-10 molecules that have been shown to affect multiple leukemias in in vitro assays with cultured leukemic cells. These assays include cell viability, apoptosis induction, and examining cell differentiation markers. Thus, within 7-10 days, the most efficient molecules are determined, which induce reprogramming of leukemic cells, which leads to inhibition of cell viability and induction of apoptosis in individual patients. Indeed, similar studies using a number of different cell lines of various leukemia types have seen differences in responses to various reprogramming agents (FIG. 14). Importantly, however, some molecules, for example ISX-9, are efficient in all tested leukemias. In total, these results further demonstrate the use of ex vivo screening of lymphoma cells from individual patients for sensitivity to treatment with combinations of EPZ and reprogramming agents (e.g. differentiation-inducers) in order to select the combination and conditions that would be most effective in individual patients.

### Example 14: The In Vivo Use of EZH1/2 Inhibition in Combination With Dexamethasone Treatment in Mouse Models of Leukemia

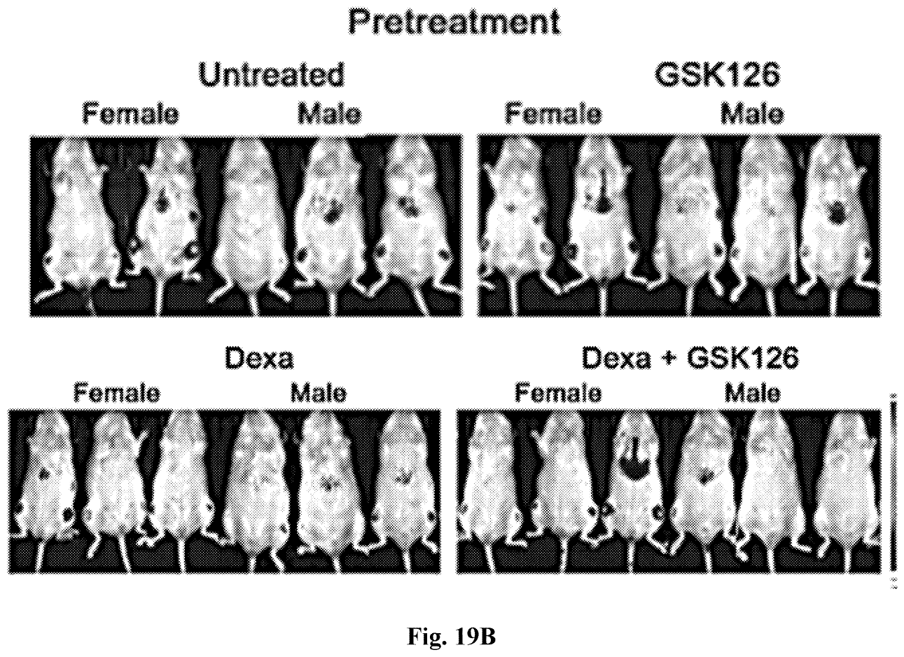

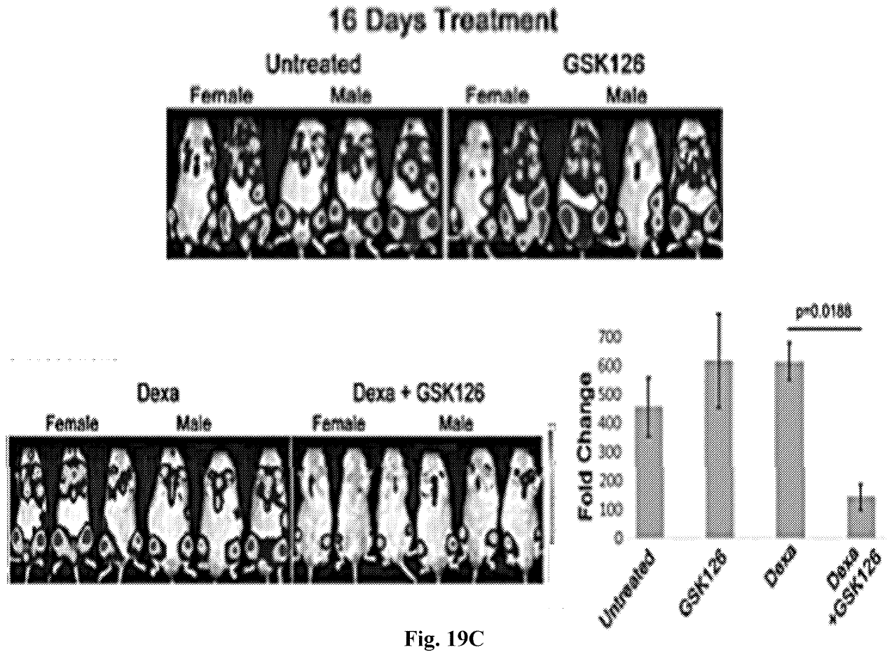

Building on previous studies using in vitro cultures of cell lines and ex vivo primary cells isolated from human patients, a series of studies was then conducted to observe the effects of EZH1/2 inhibition/Dexamethasone treatment in a mouse xenograft model of leukemia. Luciferase-expressing SEM-1 (Luc+) cells were injected into non-obese diabetic Rag1^{null}IL2rg^{null} (NRG) mice (1×10⁶/mouse), followed by treatment with the EZH2 inhibitor GSK126 or Dexamethasone, or both according to the schedule in FIG. 19A. Five days post-injection, mice were imaged and left untreated (controls), treated with GSK126 alone for 14 days (GSK126 group), or pre-treated (2 days) with GSK126 and co-treated with GSK126 and dexamethasone for 12 additional days (Dexamethasone/GSK126 group); the Dexamethasone only group was treated for 14 days, starting 7 days post SEM cell injection. (FIG. 19B) Representative serial bioluminescence images of mice acquired 5 days post-injection. (FIG. 19C) Top and bottom-left, representative serial bioluminescence images of mice acquired at the end of 16 day treatment (21 days post SEM cell injection). Bottom-right, Graphical representation of fold-changes in leukemia burden (bioluminescence quantification) of the treatment groups. While either GSK126 or Dexamethasone treatment alone demonstrated no statistical difference from untreated mice, the combination of the two compounds resulted in a dramatic and significant reduction in the growth of the cancer cells over the course of the study.

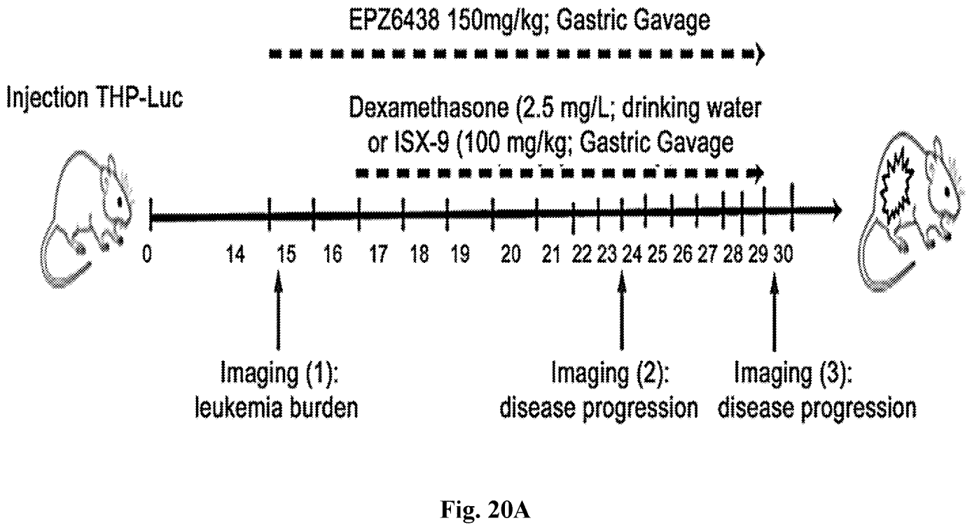

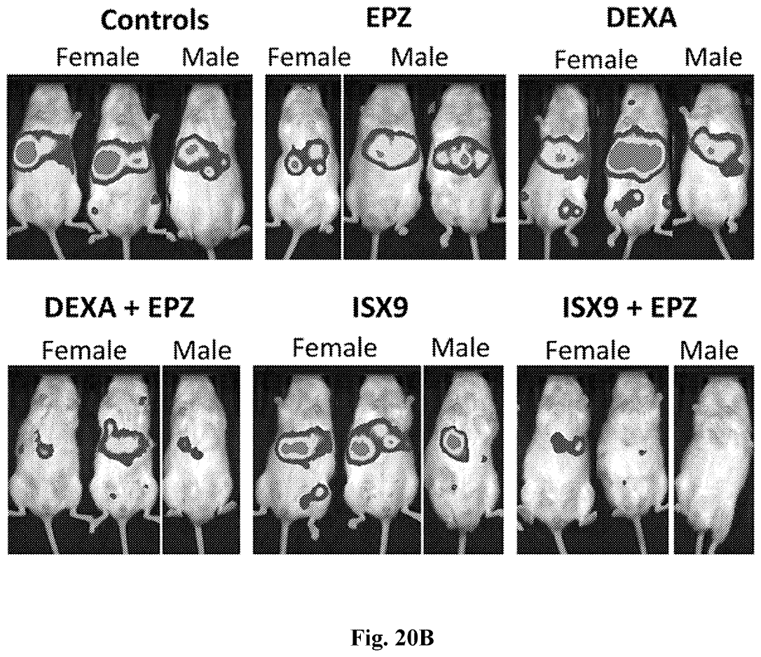

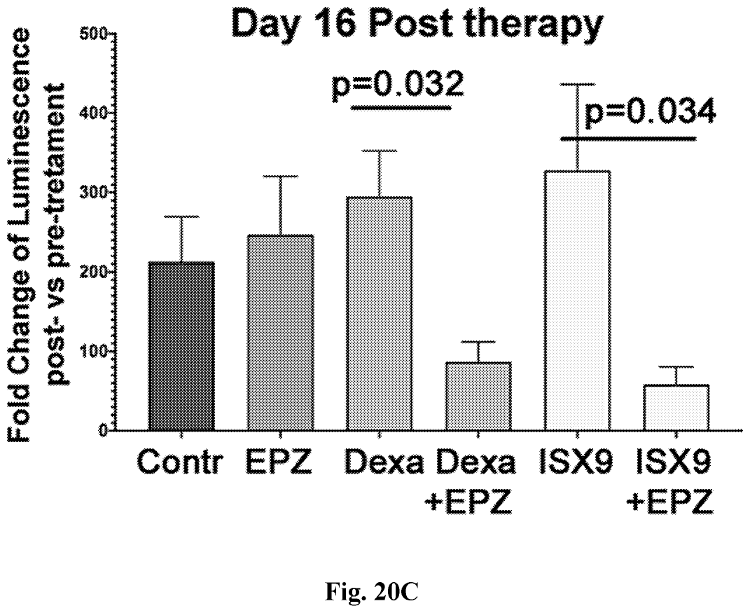

A similar study was conducted in order to examine whether the reprogramming agents ISX9 or Dexamethasone could be used in vivo in cooperation with the EPZ1/2 inhibitor EPZ to suppress AML growth in NRG mice. The treatment schedule and dosing regimen is illustrated in FIG. 20A. NRG were mice injected with THP-1 Luc+ cells, an especially aggressive AML line (1×10⁶/mouse). 14 days post-injection, mice were imaged and left untreated (Controls), treated with EPZ alone, Dexa alone and ISX9 alone for 16 days, or pre-treated (2 days) with EPZ and co-treated with Dexa and EPZ (Dexa+EPZ) or with ISX9 and EPZ (ISX9+EPZ) for 14 additional days. In FIG. 20B, representative serial bioluminescence images of mice acquired at the end of treatment (30 days post THP-1 cell injection) for all tested groups are illustrated. Quantification of the bioluminescence fold-changes in leukemia burden of the untreated and drug-treated NRG mice after treatment (30 days post injection) is represented in FIG. 20C. Similar to the previous study, treatment with EPZ, Dexamethasone, and ISX9 alone had little effect on THP-1 cell growth as compared to untreated control animals, the combination of Dexamethasone or ISX9 with EPZ resulted in a dramatic and significant delay in THP-1 cell outgrowth, with the magnitude of the delay being similar between the two reprogramming agents. Overall, these results demonstrated that the benefit of combination EZH1/2 inhibition/ reprogramming agent treatment can be observed in an in vivo setting, further suggesting the clinical utility of this strategy for the treatment of leukemias and lymphomas.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiment or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating cancer in a subject, the method comprising administering to the subject:
an effective amount of a cell cycle inhibitor,
an effective amount of a histone methyltransferase inhibitor, and
an effective amount of a reprogramming agent;
wherein the cell cycle inhibitor is an inhibitor of any one or both of CDK4 and CDK6;
wherein the histone methyltransferase inhibitor is an inhibitor of the methylation of H3K27; and
wherein the histone methyltransferase inhibitor is an inhibitor of enzyme Enhancer of Zeste Homolog 2 (EZH2);
wherein the cell cycle inhibitor, histone methyltransferase inhibitor, and reprogramming agent are administered concurrently or the cell cycle inhibitor is administered first, followed by concurrent administration of the histone methyltransferase inhibitor and reprogramming agent.

2. The method of claim 6, wherein the CDK4 and CDK6 inhibitor is selected from the group consisting of abemaciclib, palbociclib, and ribociclib.

3. The method of claim 1, wherein the EZH2 inhibitor is selected from the group consisting of tazemetostat, GSK126, 3-deazaneplanocin A, and GSK343.

4. The method of claim 1, wherein the reprogramming agent induces the terminal differentiation of hematopoietic cells.

5. The method of claim 1, wherein the reprogramming agent is selected from the group consisting of all-trans retinoic acid (ATRA), isoxazole-9 (ISX-9), dexamethasone, a vitamin D receptor agonist, and a peroxisome proliferator activated receptor (PPARγ) receptor ligand.

6. The method of claim 5, wherein the vitamin D receptor agonist is selected from the group consisting of 1,25 dihydroxy vitamin D₃, paricalcitol, and doxercalciferol.

7. The method of claim 5, wherein the PPARγ receptor ligand is a thiazolidinedione (TZD) selected from the group consisting of rosiglitazone, pioglitazone, metformin, or any combination thereof.

8. The method of claim 1, wherein the subject is a human.

9. The method of claim 1, wherein the cancer is a hematologic malignancy.

10. The method of claim 9, wherein the hematologic malignancy is selected from the group consisting of acute myelogenous leukemia, acute myeloblastic leukemia, acute myeloid leukemia, chronic myelogenous leukemia-blast crisis, and acute nonlymphocytic leukemia.

11. A method of treating cancer in a subject, comprising:
a) administering to a sample from the subject, a plurality of combinations of cell cycle inhibitors, histone methyltransferase inhibitors and reprogramming agents,
b) determining the optimal combination of cell cycle inhibitors, histone methyltransferase inhibitors, and reprogramming agents, and
c) treating the subject with the optimal combination of cell cycle inhibitors, histone methyltransferase inhibitors, and reprogramming agents thereby treating the cancer;
wherein the histone methyltransferase inhibitor is an inhibitor of the methylation of H3K27;
wherein the histone methyltransferase inhibitor is an inhibitor of enzyme Enhancer of Zeste Homolog 2 (EZH2).

12. The method of claim 11, wherein the histone methyltransferase inhibitor is an inhibitor of enzyme Enhancer of Zeste Homolog 2 (EZH2).

13. The method of claim 12, wherein the EZH2 inhibitor is selected from the group consisting of tazemetostat, GSK126, 3-deazaneplanocin A, and GSK343.

14. The method of claim 11, wherein the reprogramming agent induces the terminal differentiation of hematopoietic cells.

15. The method of claim 11, wherein the reprogramming agent is selected from the group consisting of all-trans retinoic acid (ATRA), isoxazole-9 (ISX-9), dexamethasone, a vitamin D receptor agonist, and a peroxisome proliferator activated receptor (PPARγ) receptor ligand.

16. The method of claim 15, wherein the vitamin D receptor agonist is selected from the group consisting of 1,25 dihydroxy vitamin D₃, paricalcitol, and doxercalciferol.

17. The method of claim 15, wherein the PPARγ receptor ligand is a thiazolidinedione (TZD) and is selected from the group consisting of rosiglitazone, pioglitazone, metformin, or any combination thereof.

18. The method of claim 11, wherein the subject is a human.

19. The method of claim 11, wherein the cancer is a hematologic malignancy.

20. The method of claim 19 wherein the hematologic malignancy is selected from the group consisting of acute myelogenous leukemia, acute myeloblastic leukemia, acute myeloid leukemia, chronic myelogenous leukemia-blast crisis, and acute nonlymphocytic leukemia.
